(19) United States
(12) Patent Application Publication
(10) Pub. No.: US 2010/0291551 A1
(43) Pub. Date: Nov. 18, 2010

(54) GENEMAP OF THE HUMAN ASSOCIATED WITH CROHN'S DISEASE

(75) Inventor: Abdelmajid Belouchi, Ville-St-Laurent (CA)

Correspondence Address: DOWELL & DOWELL P.C., 103 Oronoco St., Suite 220, Alexandria, VA, 22314, US

(73) Assignee: GENIZON BIOSCIENCES INC., Ville St-Laurent, Quebec (CA)

(21) Appl. No.: 12/312,580

(22) PCT Filed: Nov. 16, 2007
(86) PCT No.: PCT/US07/84977

§ 371 Date: Mar. 18, 2010

Related U.S. Application Data
(60) Provisional application No. 60/859,546, filed on Nov. 17, 2006.

(51) Int. Cl. C12Q 1/68

(52) U.S. Cl. 435/6

(57) ABSTRACT

The present invention relates to the selection of a set of polymorphism markers for use in genome wide association studies based on linkage disequilibrium mapping. In particular, the invention relates to the fields of pharmacogenomics, diagnostics, patient therapy and the use of genetic haplotype information to predict an individual's susceptibility to IBD (e.g. Crohn's Disease) and/or their response to a particular drug or drugs.

### PRIORITY

This application claims priority to U.S. Provisional Application No. 60/859,546, filed Nov. 17, 2006, which is hereby incorporated by reference in its entirety.

### FIELD OF THE INVENTION

The invention relates to the field of genomics and genetics, including genome analysis and the study of DNA variations. In particular, the invention relates to the fields of pharmacogenomics, diagnostics, patient therapy and the use of genetic haplotype information to predict an individual's susceptibility to IBD (e.g. Crohn's disease) and/or their response to a particular drug or drugs, so that drugs tailored to genetic differences of population groups may be developed and/or administered to the appropriate population.

The invention also relates to a GeneMap for IBD (e.g. Crohn's disease), which links variations in DNA (including both genic and non-genic regions) to an individual's susceptibility to IBD (e.g. Crohn's disease) and/or response to a particular drug or drugs. The invention further relates to the genes disclosed in the GeneMap (see Tables 2-4), which is related to methods and reagents for detection of an individual's increased or decreased risk for IBD (e.g. Crohn's disease) and related sub-phenotypes, by identifying at least one polymorphism in one or a combination of the genes from the GeneMap. Also related are the candidate regions identified in Table 1, which are associated with IBD (e.g. Crohn's disease). In addition, the invention further relates to nucleotide sequences of those genes including genomic DNA sequences, DNA sequences, single nucleotide polymorphisms (SNPs), other types of polymorphisms (insertions, deletions, microsatellites), alleles and haplotypes (see Sequence Listing and Tables 5.1, 5.2, 6.2 and 6.3).

The invention further relates to isolated nucleic acids comprising these nucleotide sequences and isolated polypeptides or peptides encoded thereby. Also related are expression vectors and host cells comprising the disclosed nucleic acids or fragments thereof, as well as antibodies that bind to the encoded polypeptides or peptides.

The present invention further relates to ligands that modulate the activity of the disclosed genes or gene products. In addition, the invention relates to diagnostics and therapeutics for IBD (e.g. Crohn's disease) disease, utilizing the disclosed nucleic acids, polymorphisms, chromosomal regions, GeneMaps, polypeptides or peptides, antibodies and/or ligands and small molecules that activate or repress relevant signaling events.

### BACKGROUND OF THE INVENTION

Inflammatory bowel disease (IBD) is a collective term used to describe two intestinal disorders whose etiology is not completely understood: ex: Crohn's disease (CD) and ulcerative colitis (UC). The course and prognosis of IBD, which occurs worldwide and afflicts several million people, varies widely. Onset of IBD is predominant in young adulthood. Symptoms of IBD include abdominal cramps and pain, diarrhea, weight loss and intestinal bleeding. Anemia and weight loss are also common signs of IBD. Between 10% and 15% of people with IBD require surgery over a ten-year period. Patients with IBD are also at increased risk for the development of intestinal cancer. These diseases are accompanied by a high frequency of psychological symptoms, including anxiety and depression.

There are common features in many of the later stages of IBD. Inflammation at the disease site/target organ is typically present, caused by the release of inflammatory (also termed “proinflammatory”) cytokines by T cells and by other cells that contribute to the activation steps and effector pathways of immune/inflammatory processes. The current consensus opinion regarding the pathogenesis of IBD centers on the role of genetically determined dysregulation in the host immune response toward the resident bacterial flora.

UC involves the rectum and spreads proximally to contiguous portions or to the entire colon. Disease activity is usually intermittent, with relapses and periods of quiescence. The sigmoidoscopic or colonoscopic picture is characteristic. In mild disease, the colonic mucosa appears hyperemic and granular. In more severe disease, tiny punctuate ulcers are present and the mucosa is characteristically friable and may bleed spontaneously. Histologically, the inflammatory cell infiltrate in active disease usually includes neutrophils, often invading crypts as well as being associated with epithelial damage and crypt distortion. An increased number of lymphocytes in the lamina propria and basal plasmacytosis are usually present. Between 500,000 and 700,000 patients suffer from UC in the United States. Extra-colonic manifestations of UC include arthritis, uveitis, aphthous stomatitis, pyoderma gangrenosum, and erythema nodosum. Initial therapy for patients with mild to moderate disease is usually an aminosalicylate. In controlled trials, disease improvement by various criteria occurred in up to 30% of subjects in the placebo groups; thus, no specific treatment may be an option for patients with very mild disease. In patients with active UC who do not respond to standard 5-ASA treatment and in those with more severe disease, oral corticosteroids have been the mainstay of acute symptomatic therapy. However, corticosteroids are not effective in long-term maintenance of remission in patients with UC given that their use is associated with significant toxicity over time. Although the pathogenesis of UC is not fully understood, there is increasing evidence that UC may be an autoimmune disorder, with B cells playing a role in disease pathophysiology. B cells, as well as T cells, are present in basal lymphoid aggregates, a histopathologic feature considered indicative of UC and seen in histologic sections from patients with active UC. Whereas mucosal inflammation in UC is thought to be driven by activated T cells, these patients have a T-helper-2 (Th2) cytokine expression pattern profile. As. Th2 cytokines classically drive B-cell immune responses and antibody production, a central role for B cell may be postulated in UC.

Crohn's disease is an Inflammatory Bowel Disease (IBD) in which inflammation extends beyond the inner gut lining and penetrates deeper layers of the intestinal wall of any part of the digestive system (esophagus, stomach, small intestine, large intestine, and/or anus). Crohn's disease is a chronic, lifelong disease which can cause painful, often life altering symptoms including diarrhea, cramping and rectal bleeding. Crohn's disease occurs most frequently in the industrialized world and the typical age of onset falls into two distinct ranges, 15 to 30 years of age and 60 to 80 years of age. The highest mortality is during the first years of disease, and in cases where the disease symptoms are long lasting, an increased risk of colon cancer is observed. Crohn's disease presently accounts for approximately two thirds of IBD-related physician visits and hospitalizations, and 50 to 80% of Crohn's disease patients eventually require surgical treatment. Development of Crohn's disease is influenced by environmental and host specific factors, together with “exogenous biological factors” such as constituents of the intestinal flora (the naturally occurring bacteria found in the intestine). It is believed that in genetically predisposed individuals, exogenous factors such as infectious agents, and host-specific characteristics such as intestinal barrier function and/or blood supply, combine with specific environmental factors to cause a chronic state of improperly regulated immune system function. In this hypothetical model, microorganisms trigger an immune response in the intestine, and in susceptible individuals, this immune response is not turned off when the microorganism is cleared from the body. The chronically “turned on” immune response causes damage to the intestine resulting in the symptoms of Crohn's disease.

Current treatments for Crohn's disease are primarily aimed at reducing symptoms by suppressing inflammation and do not address the root, cause of the disease. Despite a preponderance of evidence showing inheritance of a risk for Crohn's disease through epidemiological studies and genome wide linkage analyses, the genes affecting Crohn's disease have yet to be discovered (Hugot J P, and Thomas G., 1998). There is a need in the art for identifying specific genes related to Crohn's disease to enable the development of therapeutics that address the causes of the disease rather than relieving its symptoms. The failure in past studies to identify causative genes in complex diseases, such as Crohn's disease, has been due to the lack of appropriate methods to detect a sufficient number of variations in genomic DNA samples (markers), the insufficient quantity of necessary markers available, and the number of needed individuals to enable such a study.

Unfortunately, new therapies for IBD are few, and both diagnosis and treatment have been hampered by a lack of detailed knowledge of the etiology. Despite the progress noted above, there remains a need in the art for new and improved methods for treating this debilitating group of diseases, and the present inventors have made a significant step forward with the invention disclosed herein. The invention includes a method for treating IBD and inflammatory diseases in a subject, comprising administering to a subject in need of such treatment an effective amount of a pharmaceutical composition that comprises (a) an compound that inhibits inflammation; and (b) a pharmaceutically acceptable carrier, thereby treating the disease.

The present invention also relates specifically to a set of IBD (e.g. Crohn's disease) causing genes (GeneMap) and targets which present attractive points of therapeutic intervention and diagnostics.

In view of the foregoing, identifying susceptibility genes associated with IBD (e.g. Crohn's disease) and their respective biochemical pathways will facilitate the identification of diagnostic markers as well as novel targets for improved therapeutics. It will also improve the quality of life for those afflicted by this disease and will reduce the economic costs of these afflictions at the individual and societal level. The identification of those genetic markers would provide the basis for novel genetic tests and eliminate or reduce the therapeutic methods currently used. The identification of those genetic markers will also provide the development of effective therapeutic intervention for the battery of laboratory, psychological and clinical evaluations typically required to diagnose IBD (e.g. Crohn's disease). The present invention satisfies this need.

### SEQUENCE LISTING AND TABLES

The Sequence Listing and Tables filed concurrently herewith are part of the instant application, and are hereby incorporated by reference in their entireties.

### Definitions

Throughout the description of the present invention, several terms are used that are specific to the science of this field. For the sake of clarity and to avoid any misunderstanding, these definitions are provided to aid in the understanding of the specification and claims.

Allele: One of a pair, or series, of forms of a gene or non-genic region that occur at a given locus in a chromosome. Alleles are symbolized with the same basic symbol (e.g., B for dominant and b for recessive; B1, B2, Bn for n additive alleles at a locus). In a normal diploid cell there are two alleles of any one gene (one from each parent), which occupy the same relative position (locus) on homologous chromosomes. Within a population there may be more than two alleles of a gene. See multiple alleles. SNPs also have alleles, i.e., the two (or more) nucleotides that characterize the SNP.

Amplification of nucleic acids: refers to methods such as polymerase chain reaction (PCR), ligation amplification (or ligase chain reaction, LCR) and amplification methods based on the use of Q-beta replicase. These methods are well known in the art and are described, for example, in U.S. Pat. Nos. 4,683,195 and 4,683,202. Reagents and hardware for conducting PCR are commercially available. Primers useful for amplifying sequences from the disorder region are preferably complementary to, and preferably hybridize specifically to, sequences in the disorder region or in regions that flank a target region therein. Genes from Tables 2-4 generated by amplification may be sequenced directly. Alternatively, the amplified sequence(s) may be cloned prior to sequence analysis.

Antigenic component: is a moiety that binds to its specific antibody with sufficiently high affinity to form a detectable antigen-antibody complex.

Antibodies: refer to polyclonal and/or monoclonal antibodies and fragments thereof, and immunologic binding equivalents thereof, that can bind to proteins and fragments thereof or to nucleic acid sequences from the disorder region, particularly from the disorder gene products or a portion thereof. The term antibody is used both to refer to a homogeneous molecular entity, or a mixture such as a serum product made up of a plurality of different molecular entities. Proteins may be prepared synthetically in a protein synthesizer and coupled to a carrier molecule and injected over several months into rabbits. Rabbit sera are tested for immunoreactivity to the protein or fragment. Monoclonal antibodies may be made by injecting mice with the proteins, or fragments thereof. Monoclonal antibodies can be screened by ELISA and tested for specific immunoreactivity with protein or fragments thereof (Harlow et al. 1988, Antibodies: A Laboratory Manual, Cold Spring Harbor Laboratory, Cold Spring Harbor, N.Y.). These antibodies will be useful in developing assays as well as therapeutics.

Associated allele: refers to an allele at a polymorphic locus that is associated with a particular phenotype of interest, e.g., a predisposition to a disorder or a particular drug response.

cDNA: refers to complementary or copy DNA produced from an RNA template by the action of RNA-dependent DNA polymerase (reverse transcriptase). Thus, a cDNA clone means a duplex DNA sequence complementary to an RNA molecule of interest, included in a cloning vector or PCR amplified. This term includes genes from which the intervening sequences have been removed.

cDNA library: refers to a collection of recombinant DNA molecules containing cDNA inserts that together comprise essentially all of the expressed genes of an organism or tissue. A cDNA library can be prepared by methods known to one skilled in the art (see, e.g., Cowell and Austin, 1997, “DNA Library Protocols,” Methods in Molecular Biology). Generally, RNA is first isolated from the cells of the desired organism, and the RNA is used to prepare cDNA molecules.

Cloning: refers to the use of recombinant DNA techniques to insert a particular gene or other DNA sequence into a vector molecule. In order to successfully clone a desired gene, it is necessary to use methods for generating DNA fragments, for joining the fragments to vector molecules, for introducing the composite DNA molecule into a host cell in which it can replicate, and for selecting the clone having the target gene from amongst the recipient host cells.

Cloning vector: refers to a plasmid or phage DNA or other DNA molecule that is able to replicate in a host cell. The cloning vector is typically characterized by one or more endonuclease recognition sites at which such DNA sequences may be cleaved in a determinable fashion without loss of an essential biological function of the DNA, and which may contain a selectable marker suitable for use in the identification of cells containing the vector.

Coding sequence or a protein-coding sequence: is a polynucleotide sequence capable of being transcribed into mRNA and/or capable of being translated into a polypeptide or peptide. The boundaries of the coding sequence are typically determined by a translation start codon at the 5′-terminus and a translation stop codon at the 3′-terminus.

Complement of a nucleic acid sequence: refers to the antisense sequence that participates in Watson-Crick base-pairing with the original sequence.

Disorder region: refers to the portions of the human chromosomes displayed in Table 1 bounded by the markers from Tables 2-6.

Disorder-associated nucleic acid or polypeptide sequence: refers to a nucleic acid sequence that maps to region of Table 1 or the polypeptides encoded therein (Tables 2-4, nucleic acids, and polypeptides). For nucleic acids, this encompasses sequences that are identical or complementary to the gene sequences from Tables 2-4, as well as sequence-conservative, function-conservative, and non-conservative variants thereof. For polypeptides, this encompasses sequences that are identical to the polypeptide, as well as function-conservative and non-conservative variants thereof. Included are the alleles of naturally-occurring polymorphisms causative of IBD (e.g. Crohn's disease) such as, but not limited to, alleles that cause altered expression of genes of Tables 2-4 and alleles that cause altered protein levels or stability (e.g., decreased levels, increased levels, expression in an inappropriate tissue type, increased stability, and decreased stability).

Expression vector: refers to a vehicle or plasmid that is capable of expressing a gene that has been cloned into it, after transformation or integration in a host cell. The cloned gene is usually placed under the control of (i.e., operably linked to) a regulatory sequence.

Function-conservative variants: are those in which a change in one or more nucleotides in a given codon position results in a polypeptide sequence in which a given amino acid residue in the polypeptide has been replaced by a conservative amino acid substitution. Function-conservative variants also include analogs of a given polypeptide and any polypeptides that have the ability to elicit antibodies specific to a designated polypeptide.

Founder population: Also a population isolate, this is a large number of people who have mostly descended, in genetic isolation from other populations, from a much smaller number of people who lived many generations ago.

Gene: Refers to a DNA sequence that encodes through its template or messenger RNA a sequence of amino acids characteristic of a specific peptide, polypeptide, or protein. The term “gene” also refers to a DNA sequence that encodes an RNA product. The term gene as used herein with reference to genomic DNA includes intervening, non-coding regions, as well as regulatory regions, and can include 5′ and 3′ ends. A gene sequence is wild-type if such sequence is usually found in individuals unaffected by the disorder or condition of interest. However, environmental factors and other genes can also play an important role in the ultimate determination of the disorder. In the context of complex disorders involving multiple genes (oligogenic disorder), the wild type, or normal sequence can also be associated with a measurable risk or susceptibility, receiving its reference status based on its frequency in the general population.

GeneMaps: are defined as groups of gene(s) that are directly or indirectly involved in at least one phenotype of a disorder (some non-limiting example of GeneMaps comprises varius combinations of genes from Tables 2-4). As such, GeneMaps enable the development of synergistic diagnostic products, creating “theranostics”.

Genotype: Set of alleles at a specified locus or loci.

Haplotype: The allelic pattern of a group of (usually contiguous) DNA markers or other polymorphic loci along an individual chromosome or double helical DNA segment. Haplotypes identify individual chromosomes or chromosome segments. The presence of shared haplotype patterns among a group of individuals implies that the locus defined by the haplotype has been inherited, identical by descent (IBD), from a common ancestor. Detection of identical by descent haplotypes is the basis of linkage disequilibrium (LD) mapping. Haplotypes are broken down through the generations by recombination and mutation. In some instances, a specific allele or haplotype may be associated with susceptibility to a disorder or condition of interest, e.g., CROHN disease. In other instances, an allele or haplotype may be associated with a decrease in susceptibility to a disorder or condition of interest, i.e., a protective sequence.

Host: includes prokaryotes and eukaryotes. The term includes an organism or cell that is the recipient of an expression vector (e.g., autonomously replicating or integrating vector).

Hybridizable: nucleic acids are hybridizable to each other when at least one strand of the nucleic acid can anneal to another nucleic acid strand under defined stringency conditions. In some embodiments, hybridization requires that the two nucleic acids contain at least 10 substantially complementary nucleotides; depending on the stringency of hybridization, however, mismatches may be tolerated. The appropriate stringency for hybridizing nucleic acids depends on the length of the nucleic acids and the degree of complementarity, and can be determined in accordance with the methods described herein.

Identity by descent (IBD): Identity among DNA sequences for different individuals that is due to the fact that they have all been inherited from a common ancestor. LD mapping identifies IBD haplotypes as the likely location of disorder genes shared by a group of patients.

Identity: as known in the art, is a relationship between two or more polypeptide sequences or two or more polynucleotide sequences, as determined by comparing the sequences. In the art, identity also means the degree of sequence relatedness between polypeptide or polynucleotide sequences, as the case may be, as determined by the match between strings of such sequences. Identity and similarity can be readily calculated by known methods, including but not limited to those described in A. M. Lesk (ed), 1988, Computational Molecular Biology, Oxford University Press, NY; D. W. Smith (ed), 1993, Biocomputing. Informatics and Genome Projects, Academic Press, NY; A. M. Griffin and H. G. Griffin, H. G (eds), 1994, ComputerAnalysis of Sequence Data, Part 1, Humana Press, NJ; G. von Heinje, 1987, Sequence Analysis in Molecular Biology, Academic Press; and M. Gribskov and J. Devereux (eds), 1991, Sequence Analysis Primer, M Stockton Press, NY; H. Carillo and D. Lipman, 1988, SIAM J. Applied Math., 48:1073.

Immunogenic component: is a moiety that is capable of eliciting a humoral and/or cellular immune response in a host animal.

Isolated nucleic acids: are nucleic acids separated away from other components (e.g., DNA, RNA, and protein) with which they are associated (e.g., as obtained from cells, chemical synthesis systems, or phage or nucleic acid libraries). Isolated nucleic acids are at least 60% free, preferably 75% free, and most preferably 90% free from other associated components. In accordance with the present invention, isolated nucleic acids can be obtained by methods described herein, or other established methods, including isolation from natural sources (e.g., cells, tissues, or organs), chemical synthesis, recombinant methods, combinations of recombinant and chemical methods, and library screening methods.

Isolated polypeptides or peptides: are those that are separated from other components (e.g., DNA, RNA, and other polypeptides or peptides) with which they are associated (e.g., as obtained from cells, translation systems, or chemical synthesis systems). In a preferred embodiment, isolated polypeptides or peptides are at least 10% pure; more preferably, 80% or 90% pure. Isolated polypeptides and peptides include those obtained by methods described herein, or other established methods, including isolation from natural sources (e.g., cells, tissues, or organs), chemical synthesis, recombinant methods, or combinations of recombinant and chemical methods. Proteins or polypeptides referred to herein as recombinant are proteins or polypeptides produced by the expression of recombinant nucleic acids. A portion as used herein with regard to a protein or polypeptide, refers to fragments of that protein or polypeptide. The fragments can range in size from 5 amino acid residues to all but one residue of the entire protein sequence. Thus, a portion or fragment can be at least 5, 5-50, 50-100, 100-200, 200-400, 400-800, or more consecutive amino acid residues of a protein or polypeptide.

Linkage disequilibrium (LD): the situation in which the alleles for two or more loci do not occur together in individuals sampled from a population at frequencies predicted by the product of their individual allele frequencies. In other words, markers that are in LD do not follow Mendel's second law of independent random segregation. LD can be caused by any of several demographic or population artifacts as well as by the presence of genetic linkage between markers. However, when these artifacts are controlled and eliminated as sources of LD, then LD results directly from the fact that the loci involved are located close to each other on the same chromosome so that specific combinations of alleles for different markers (haplotypes) are inherited together. Markers that are in high LD can be assumed to be located near each other and a marker or haplotype that is in high LD with a genetic trait can be assumed to be located near the gene that affects that trait. The physical proximity of markers can be measured in family studies where it is called linkage or in population studies where it is called linkage disequilibrium.

LD mapping: population based gene mapping, which locates disorder genes by identifying regions of the genome where haplotypes or marker variation patterns are shared statistically more frequently among disorder patients compared to healthy controls. This method is based upon the assumption that many of the patients will have inherited an allele associated with the disorder from a common ancestor (IBD), and that this allele will be in LD with the disorder gene.

Locus: a specific position along a chromosome or DNA sequence. Depending upon context, a locus could be a gene, a marker, a chromosomal band or a specific sequence of one or more nucleotides.

Minor allele frequency (MAF): the population frequency of one of the alleles for a given polymorphism, which is equal or less than 50%. The sum of the MAF and the Major allele frequency equals one.

Markers: an identifiable DNA sequence that is variable (polymorphic) for different individuals within a population. These sequences facilitate the study of inheritance of a trait or a gene. Such markers are used in mapping the order of genes along chromosomes and in following the inheritance of particular genes; genes closely linked to the marker or in LD with the marker will generally be inherited with it. Two types of markers are commonly used in genetic analysis, microsatellites and SNPs.

Microsatellite: DNA of eukaryotic cells comprising a repetitive, short sequence of DNA that is present as tandem repeats and in highly variable copy number, flanked by sequences unique to that locus.

Mutant sequence: if it differs from one or more wild-type sequences. For example, a nucleic acid from a gene listed in Tables 2-4 containing a particular allele of a single nucleotide polymorphism may be a mutant sequence. In some cases, the individual carrying this allele has increased susceptibility toward the disorder or condition of interest. In other cases, the mutant sequence might also refer to an allele that decreases the susceptibility toward a disorder or condition of interest and thus acts in a protective manner. The term mutation may also be used to describe a specific allele of a polymorphic locus.

Non-conservative variants: are those in which a change in one or more nucleotides in a given codon position results in a polypeptide sequence in which a given amino acid residue in a polypeptide has been replaced by a non-conservative amino acid substitution. Non-conservative variants also include polypeptides comprising non-conservative amino acid substitutions.

Nucleic acid or polynucleotide: purine- and pyrimidine-containing polymers of any length, either polyribonucleotides or polydeoxyribonucleotide or mixed polyribo polydeoxyribonucleotides. This includes single- and double-stranded molecules, i.e., DNA-DNA, DNA-RNA and RNA-RNA hybrids, as well as protein nucleic acids (PNA) formed by conjugating bases to an amino acid backbone. This also includes nucleic acids containing modified bases.

Nucleotide: a nucleotide, the unit of a DNA molecule, is composed of a base, a 2′-deoxyribose and phosphate ester(s) attached at the 5′ carbon of the deoxyribose. For its incorporation in DNA, the nucleotide needs to possess three phosphate esters but it is converted into a monoester in the process.

Operably linked: means that the promoter controls the initiation of expression of the gene. A promoter is operably linked to a sequence of proximal DNA if upon introduction into a host cell the promoter determines the transcription of the proximal DNA sequence(s) into one or more species of RNA. A promoter is operably linked to a DNA sequence if the promoter is capable of initiating transcription of that DNA sequence.

Ortholog: denotes a gene or polypeptide obtained from one species that has homology to an analogous gene or polypeptide from a different species.

Paralog: denotes a gene or polypeptide obtained from a given species that has homology to a distinct gene or polypeptide from that same species.

Phenotype: any visible, detectable or otherwise measurable property of an organism such as symptoms of, or susceptibility to, a disorder.

Polymorphism: occurrence of two or more alternative genomic sequences or alleles between or among different genomes or individuals at a single locus. A polymorphic site thus refers specifically to the locus at which the variation occurs. In some cases, an individual carrying a particular allele of a polymorphism has an increased or decreased susceptibility toward a disorder or condition of interest.

Portion and fragment: are synonymous. A portion as used with regard to a nucleic acid or polynucleotide refers to fragments of that nucleic acid or polynucleotide. The fragments can range in size from 8 nucleotides to all but one nucleotide of the entire gene sequence. Preferably, the fragments are at least about 8 to about 10 nucleotides in length; at least about 12 nucleotides in length; at least about 15 to about 20 nucleotides in length; at least about 25 nucleotides in length; or at least about 35 to about 55 nucleotides in length.

Probe or primer: refers to a nucleic acid or oligonucleotide that forms a hybrid structure with a sequence in a target region of a nucleic acid due to complementarity of the probe or primer sequence to at least one portion of the target region sequence.

Protein and polypeptide: are synonymous. Peptides are defined as fragments or portions of polypeptides, preferably fragments or portions having at least one functional activity (e.g., proteolysis, adhesion, fusion, antigenic, or intracellular activity) as the complete polypeptide sequence.

Recombinant nucleic acids: nucleic acids which have been produced by recombinant DNA methodology, including those nucleic acids that are generated by procedures which rely upon a method of artificial replication, such as the polymerase chain reaction (PCR) and/or cloning into a vector using restriction enzymes. Portions of recombinant nucleic acids which code for polypeptides can be identified and isolated by, for example, the method of M. Jasin et al., U.S. Pat. No. 4,952,501.

Regulatory sequence: refers to a nucleic acid sequence that controls or regulates expression of structural genes when operably linked to those genes. These include, for example, the lac systems, the trp system, major operator and promoter regions of the phage lambda, the control region of fd coat protein and other sequences known to control the expression of genes in prokaryotic or eukaryotic cells. Regulatory sequences will vary depending on whether the vector is designed to express the operably linked gene in a prokaryotic or eukaryotic host, and may contain transcriptional elements such as enhancer elements, termination sequences, tissue-specificity elements and/or translational initiation and termination sites.

Sample: as used herein refers to a biological sample, such as, for example, tissue or fluid isolated from an individual or animal (including, without limitation, plasma, serum, cerebrospinal fluid, lymph, tears, nails, hair, saliva, milk, pus, and tissue exudates and secretions) or from in vitro cell culture-constituents, as well as samples obtained from, for example, a laboratory procedure.

Single nucleotide polymorphism (SNP): variation of a single nucleotide. This includes the replacement of one nucleotide by another and deletion or insertion of a single nucleotide. Typically, SNPs are biallelic markers although tri- and tetra-allelic markers also exist. For example, SNP A\C may comprise allele C or allele A (Tables 5.1, 5.2, 6.2 and 6.3). Thus, a nucleic acid molecule comprising SNP A\C may include a C or A at the polymorphic position. For clarity purposes, an ambiguity code is used in Tables 5.1, 5.2, 6.2 and 6.3 and the sequence listing, to represent the variations. For a combination of SNPs, the term “haplotype” is used, e.g. the genotype of the SNPs in a single DNA strand that are linked to one another. In certain embodiments, the term “haplotype” is used to describe a combination of SNP alleles, e.g., the alleles of the SNPs found together on a single DNA molecule. In specific embodiments, the SNPs in a haplotype are in linkage disequilibrium with one another.

Sequence-conservative: variants are those in which a change of one or more nucleotides in a given codon position results in no alteration in the amino acid encoded at that position (i.e., silent mutation).

Substantially homologous: a nucleic acid or fragment thereof is substantially homologous to another if, when optimally aligned (with appropriate nucleotide insertions and/or deletions) with the other nucleic acid (or its complementary strand), there is nucleotide sequence identity in at least 60% of the nucleotide bases, usually at least 70%, more usually at least 80%, preferably at least 90%, and more preferably at least 95-98% of the nucleotide bases. Alternatively, substantial homology exists when a nucleic acid or fragment thereof will hybridize, under selective hybridization conditions, to another nucleic acid (or a complementary strand thereof). Selectivity of hybridization exists when hybridization which is substantially more selective than total lack of specificity occurs. Typically, selective hybridization will occur when there is at least about 55% sequence identity over a stretch of at least about nine or more nucleotides, preferably at least about 65%, more preferably at least about 75%, and most preferably at least about 90% (M. Kanehisa, 1984, Nucl. Acids Res. 11:203-213). The length of homology comparison, as described, may be over longer stretches, and in certain embodiments will often be over a stretch of at least 14 nucleotides, usually at least 20 nucleotides, more usually at least 24 nucleotides, typically at least 28 nucleotides, more typically at least 32 nucleotides, and preferably at least 36 or more nucleotides.

Wild-type gene from Tables 2-4: refers to the reference sequence. The wild-type gene sequences from Tables 2-4 used to identify the variants (polymorphisms, alleles, and haplotypes) described in detail herein.

Technical and scientific terms used herein have the meanings commonly understood by one of ordinary skill in the art to which the present invention pertains, unless otherwise defined. Reference is made herein to various methodologies known to those of skill in the art. Publications and other materials setting forth such known methodologies to which reference is made are incorporated herein by reference in their entireties as though set forth in full. Standard reference works setting forth the general principles of recombinant DNA technology include J. Sambrook et al., 1989, Molecular Cloning: A Laboratory Manual, 2d Ed., Cold Spring Harbor Laboratory Press, Cold Spring Harbor, N.Y.; P. B. Kaufman et al., (eds), 1995, Handbook of Molecular and Cellular Methods in Biology and Medicine, CRC Press, Boca Raton; M. J. McPherson (ed), 1991, Directed Mutagenesis: A Practical Approach, IRL Press, Oxford; J. Jones, 1992, Amino Acid and Peptide Synthesis, Oxford Science Publications, Oxford; B. M. Austen and O. M. R. Westwood, 1991, Protein Targeting and Secretion, IRL Press, Oxford; D. N Glover (ed), 1985, DNA Cloning, Volumes 1 and 11; M. J. Gait (ed), 1984, Oligonucleotide Synthesis; B. D. Hames and S. J. Higgins (eds), 1984, Nucleic Acid Hybridization; Quirke and Taylor (eds), 1991, PCR-A Practical Approach; Harries and Higgins (eds), 1984, Transcription and Translation; R. I. Freshney (ed), 1986, Animal Cell Culture; Immobilized Cells and Enzymes, 1986, IRL Press; Perbal, 1984, A Practical Guide to Molecular Cloning, J. H. Miller and M. P. Calos (eds), 1987, Gene Transfer Vectors for Mammalian Cells, Cold Spring Harbor Laboratory Press; M. J. Bishop (ed), 1998, Guide to Human Genome Computing, 2d Ed., Academic Press, San Diego, Calif.; L. F. Peruski and A. H. Peruski, 1997, The Internet and the New Biology. Tools for Genomic and Molecular Research, American Society for Microbiology, Washington, D.C. Standard reference works setting forth the general principles of immunology include S. Sell, 1996, Immunology, Immunopathology & Immunity, 5th Ed., Appleton & Lange, Publ., Stamford, Conn.; D. Male et al., 1996, Advanced Immunology, 3d Ed., Times Mirror Int'l Publishers Ltd., Publ., London; D. P. Stites and A. L Terr, 1991, Basic and Clinical Immunology, 7th Ed., Appleton & Lange, Publ., Norwalk, Conn.; and A. K. Abbas et al., 1991, Cellular and Molecular Immunology, W. B. Saunders Co., Publ., Philadelphia, Pa. Any suitable materials and/or methods known to those of skill can be utilized in carrying out the present invention; however, preferred materials and/or methods are described. Materials, reagents, and the like to which reference is made in the following description and examples are generally obtainable from commercial sources, and specific vendors are cited herein.

### DETAILED DESCRIPTION OF THE INVENTION

### Genome Wide Association Study to Construct a GeneMap for IBD

The present invention is based on the discovery of genes associated with IBD (e.g. Crohn's disease). In the preferred embodiment, disease-associated loci (candidate regions; Table 1) are identified by the statistically significant differences in allele or haplotype frequencies between the cases and the controls. For the purpose of the present invention, 378 candidate regions (Table 1) are identified.

The invention provides a method for the discovery of genes associated with IBD (e.g. Crohn's disease) and the construction of a GeneMap for IBD (e.g. Crohn's disease) in a human population, comprising the following steps (see also Example section herein):

### Step 1: Recruit Patients (Cases) and Controls

In the preferred embodiment, 382 patients diagnosed for CROHN disease along with 382 independent controls samples are recruited from the German population.

In another preferred embodiment, 500 patients diagnosed for CROHN disease along with 500 independent controls samples are recruited from the Quebec Founder population.

In another embodiment, more or less than 500 patients and controls are recruited.

In another embodiment, 500 patients diagnosed for CROHN disease along with two family members are recruited from the Quebec founder or German populations. The preferred trios recruited are parent-parent-child (PPC) trios. Trios can also be recruited as parent-child-child (PCC) trios. In another preferred embodiment, more or less than 500 trios are recruited

In yet another embodiment, the present invention is performed as a whole or partially with DNA samples from individuals of another population resource.

### Step 2: DNA Extraction and Quantitation

Any sample comprising cells or nucleic acids from patients or controls may be used. Preferred samples are those easily obtained from the patient or control. Such samples include, but are not limited to blood, peripheral lymphocytes, buccal swabs, epithelial cell swabs, nails, hair, bronchoalveolar lavage fluid, sputum, or other body fluid or tissue obtained from an individual.

In one embodiment, DNA is extracted from such samples in the quantity and quality necessary to perform the invention using conventional DNA extraction and quantitation techniques. The present invention is not linked to any DNA extraction or quantitation platform in particular.

### Step 3: Genotype the Recruited Individuals

In one embodiment, assay-specific and/or locus-specific and/or allele-specific oligonucleotides for every SNP marker of the present invention (Tables 5.1, 5.2, 6.2 and 6.3) are organized onto one or more arrays. The genotype at each SNP locus is revealed by hybridizing short PCR fragments comprising each SNP locus onto these arrays. The arrays permit a high-throughput genome wide association study using DNA samples from individuals of the Quebec Founder or the German populations. Such assay-specific and/or locus-specific and/or allele-specific oligonucleotides necessary for scoring each SNP of the present invention are preferably organized onto a solid support. Such supports can be arrayed on wafers, glass slide, beads or any other type of solid support.

In another embodiment, the assay-specific and/or locus-specific and/or allele-specific oligonucleotides are not organized onto a solid support but are still used as a whole, in panels or one by one. The present invention is therefore not linked to any genotyping platform in particular.

In another embodiment, one or more portions of the SNP maps are used to screen the whole genome, a subset of chromosomes, a chromosome, a subset of genomic regions or a single genomic region.

In the preferred embodiment, the individuals composing the cases and controls or the trios are preferably individually genotyped with at least 80,000 markers, generating at least a few million genotypes; more preferably, at least a hundred million. In another embodiment, individuals are pooled in cases and control pools for genotyping and genetic analysis.

Step 4: Exclude the Markers that Did not Pass the Quality Control of the Assay.

Preferably, the quality controls comprises, but are not limited to, the following criteria: eliminate SNPs that had a high rate of Mendelian errors (cut-off at 1% Mendelian error rate), that deviate from the Hardy-Weinberg equilibrium, that are non-polymorphic in the population or have too many missing data (cut-off at 1% missing values or higher), or simply because they are non-polymorphic in the population (cut-off between 1% and 10% minor allele frequency (MAF)).

### Step 5: Perform the Genetic Analysis on the Results Obtained Using Haplotype Information as Well as Single-Marker Association.

In the preferred embodiment, genetic analysis is performed on all the genotypes from Step 3.

In another embodiment, genetic analysis is performed on a subset of markers from Step 3 or from markers that passed the quality controls from Step 4.

In one embodiment, the genetic analysis consists of, but is not limited to features corresponding to Phase information and haplotype structures. Phase information and haplotype structures are preferably deduced from genotypes using Phasefinder. Since chromosomal assignment (phase) cannot be estimated when all trio members are heterozygous, an Expectation-Maximization (EM) algorithm may be used to resolve chromosomal assignment ambiguities after Phasefinder.

In yet another embodiment, the PL-EM algorithm (Partition-Ligation E M; Niu at al., Am. J. Hum. Genet. 70:157 (2002)) can be used to estimate haplotypes from the “genotype” data as a measured estimate of the reference allele frequency of a SNP in 15-marker windows that advance in increments of one marker across the data set. The results from such algorithms are converted into 15-marker haplotype files.

In another embodiment, the haplotype frequencies among patients are compared to those among the controls using LDSTATS, a program that assesses the association of haplotypes with the disease. Such program defines haplotypes using multi-marker windows that advance across the marker map in one-marker increments. Such windows can be 1, 3, 5, 7 or 9 markers wide, and all these window sizes are tested concurrently. Larger multi-marker haplotype windows can also be used. At each position the frequency of haplotypes in cases is compared to the frequency of haplotypes in controls. Such allele frequency differences for single marker windows can be tested using Pearson's Chi-square with any degree of freedom. Multi-allelic haplotype association can be tested using Smith's normalization of the square root of Pearson's Chi-square. Such significance of association can be reported in two ways:

The significance of association within any one haplotype window is plotted against the marker that is central to that window.

P-values of association for each specific marker are calculated as a pooled P-value across all haplotype windows in which they occur. The pooled P-value is calculated using an expected value and variance calculated using a permutation test that considers covariance between individual windows. Such pooled P-values can yield narrower regions of gene location than the window data (see Example 3 herein for details on various analysis methods, such as LDSTATS v2.0).

In another embodiment, conditional and subphenotype analyses can be performed on subsets of the original set of cases and controls using the program LDSTATS. For conditional analyses, the selection of a subset of cases and their matched controls can be based on the carrier status of cases at a gene or locus of interest.

### Step 6: SNP and DNA Polymorphism Discovery

In the preferred embodiment, all the candidate genes and regions identified in step 5 are sequenced for polymorphism identification.

In another embodiment, the entire region, including all introns, is sequenced to identify all polymorphisms.

In yet another embodiment, the candidate genes are prioritized for sequencing, and only functional gene elements (promoters, conserved noncoding sequences, exons and splice sites) are sequenced.

In yet another embodiment, previously identified polymorphisms in the candidate regions can also be used. For example, SNPs from dbSNP, or others can also be used rather than resequencing the candidate regions to identify polymorphisms.

The discovery of SNPs and DNA polymorphisms generally comprises a step consisting of determining the major haplotypes in the region to be sequenced. The preferred samples are selected according to which haplotypes contribute to the association signal observed in the region to be sequenced. The purpose is to select a set of samples that covers all the major haplotypes in the given region. Each major haplotype is preferably analyzed in at least a few individuals.

Any analytical procedure may be used to detect the presence or absence of variant nucleotides at one or more polymorphic positions of the invention. In general, the detection of allelic variation requires a mutation discrimination technique, optionally an amplification reaction and optionally a signal generation system. Any means of mutation detection or discrimination may be used. For instance, DNA sequencing, scanning methods, hybridization, extension based methods, incorporation based methods, restriction enzyme-based methods and ligation-based methods may be used in the methods of the invention.

Sequencing methods include, but are not limited to, direct sequencing, and sequencing by hybridization. Scanning methods include, but are not limited to, protein truncation test (PTT), single-strand conformation polymorphism analysis (SSCP), denaturing gradient gel electrophoresis (DGGE), temperature gradient gel electrophoresis (TGGE), cleavage, heteroduplex analysis, chemical mismatch cleavage (CMC), and enzymatic mismatch cleavage. Hybridization-based methods of detection include, but are not limited to, solid phase hybridization such as dot blots, multiple allele specific diagnostic assay (MASDA), reverse dot blots, and oligonucleotide arrays (DNA Chips). Solution phase hybridization amplification methods may also be used, such as Taqman. Extension based methods include, but are not limited to, amplification refraction mutation systems (ARMS), amplification refractory mutation systems (ALEX), and competitive oligonucleotide priming systems (COPS). Incorporation based methods include, but are not limited to, mini-sequencing and arrayed primer extension (APEX). Restriction enzyme-based detection systems include, but are not limited to, restriction site generating PCR. Lastly, ligation based detection methods include, but are not limited to, oligonucleotide ligation assays (OLA). Signal generation or detection systems that may be used in the methods of the invention include, but are not limited to, fluorescence methods such as fluorescence resonance energy transfer (FRET), fluorescence quenching, fluorescence polarization as well as other chemiluminescence, electrochemiluminescence, Raman, radioactivity, colometric methods, hybridization protection assays and mass spectrometry methods. Further amplification methods include, but are not limited to self sustained replication (SSR), nucleic acid sequence based amplification (NASBA), ligase chain reaction (LCR), strand displacement amplification (SDA) and branched DNA (B-DNA).

Sequencing can also be performed using a proprietary sequencing technology (Cantaloupe; PCT/EP2005/002870).

### Step 7: Ultrafine Mapping

This step further maps the candidate regions and genes confirmed in the IBD (e.g. Crohn's disease) in the human population.

In a preferred embodiment, the discovered SNPs and polymorphisms of step 6 are ultrafine mapped at a higher density of markers than the GWS described herein using the same technology described in step 3.

### Step 8: GeneMap Construction

The confirmed variations in DNA (including both genic and non-genic regions) are used to build a GeneMap for IBD (e.g. Crohn's disease). The gene content of this GeneMap is described in more detail below. Such GeneMap can be used for other methods of the invention comprising the diagnostic methods described herein, the susceptibility to IBD (e.g. Crohn's disease), the response to a particular drug, the efficacy of a particular drug, the screening methods described herein and the treatment methods described herein.

As is evident to one of ordinary skill in the art, all of the above steps or the steps do not need to be performed, or performed in a given order to practice or use the SNPs, genomic regions, genes, proteins, etc. in the methods of the invention.

Genes from the GeneMap

In one embodiment the GeneMap consists of genes and targets, in a variety of combinations, identified from the candidate regions listed in Table 1. In another embodiment, all genes from Tables 2-4 are present in the GeneMap. In another preferred embodiment, the GeneMap consists of a selection of genes from Tables 2-4. The genes of the invention (Tables 2-4) are arranged by candidate regions and by their chromosomal location. Such order is for the purpose of clarity and does not reflect any other criteria of selection in the association of the genes with IBD (e.g. Crohn's disease).

In one embodiment, genes identified in the GWAS and subsequent studies are evaluated using the Ingenuity Pathway Analysis application (IPA, Ingenuity systems) in order to identify direct biological interactions between these genes, and also to identify molecular regulators acting on those genes (indirect interactions) that could be also involved in IBD (e.g. Crohn's disease). The purpose of this effort is to decipher the molecules involved in contributing to IBD (e.g. Crohn's disease). These gene interaction networks are very valuable tools in the sense that they facilitate extension of the map of gene products that could represent potential drug targets for IBD (e.g. Crohn's disease).

In another embodiment, other means (such as fuctional biochemical assays and genetic assays) are used to identify the biological interactions between genes to create a GeneMap.

### Nucleic Acid Sequences

The nucleic acid sequences of the present invention may be derived from a variety of sources including DNA, cDNA, synthetic DNA, synthetic RNA, derivatives, mimetics or combinations thereof. Such sequences may comprise genomic DNA, which may or may not include naturally occurring introns, genic regions, nongenic regions, and regulatory regions. Moreover, such genomic DNA may be obtained in association with promoter regions or poly (A) sequences. The sequences, genomic DNA, or cDNA may be obtained in any of several ways. Genomic DNA can be extracted and purified from suitable cells by means well known in the art. Alternatively, mRNA can be isolated from a cell and used to produce cDNA by reverse transcription or other means. The nucleic acids described herein are used in certain embodiments of the methods of the present invention for production of RNA, proteins or polypeptides, through incorporation into cells, tissues, or organisms. In one embodiment, DNA containing all or part of the coding sequence for the genes described in Tables 2-4, or the SNP markers described in Tables 5.1, 5.2, 6.2 and 6.3, is incorporated into a vector for expression of the encoded polypeptide in suitable host cells. The invention also comprises the use of the nucleotide sequence of the nucleic acids of this invention to identify DNA probes for the genes described in Tables 2-4 or the SNP markers described in Tables 5.1, 5.2, 6.2 and 6.3, PCR primers to amplify the genes described in Tables 2-4 or the SNP markers described in Tables 5.1, 5.2, 6.2 and 6.3, nucleotide polymorphisms in the genes described in Tables 2-4, and regulatory elements of the genes described in Tables 2-4. The nucleic acids of the present invention find use as primers and templates for the recombinant production of IBD (e.g. Crohn's disease)—associated peptides or polypeptides, for chromosome and gene mapping, to provide antisense sequences, for tissue distribution studies, to locate and obtain full length genes, to identify and obtain homologous sequences (wild-type and mutants), and in diagnostic applications.

### Antisense Oligonucleotides

In a particular embodiment of the invention, an antisense nucleic acid or oligonucleotide is wholly or partially complementary to, and can hybridize with, a target nucleic acid (either DNA or RNA) having the sequence from any Tables of the invention as provided herewith (e.g. Tables 1, 2, 3, 4, 5.2). For example, an antisense nucleic acid or oligonucleotide comprising 16 nucleotides can be sufficient to inhibit expression of at least one gene from Tables 2-4. Alternatively, an antisense nucleic acid or oligonucleotide can be complementary to 5′ or 3′ untranslated regions, or can overlap the translation initiation codon (5′ untranslated and translated regions) of at least one gene from Tables 2-4, or its functional equivalent. In another embodiment, the antisense nucleic acid is wholly or partially complementary to, and can hybridize with, a target nucleic acid that encodes a polypeptide from a gene described in Tables 2-4.

In addition, oligonucleotides can be constructed which will bind to duplex nucleic acid (i.e., DNA:DNA or DNA:RNA), to form a stable triple helix containing or triplex nucleic acid. Such triplex oligonucleotides can inhibit transcription and/or expression of a gene from Tables 2-4, or its functional equivalent (M. D. Frank-Kamenetskii et al., 1995). Triplex oligonucleotides are constructed using the basepairing rules of triple helix formation and the nucleotide sequence of the genes described in Tables 2-4.

The present invention encompasses methods of using oligonucleotides in antisense inhibition of the function of the genes from Tables 2-4. In the context of this invention, the term “oligonucleotide” refers to naturally-occurring species or synthetic species formed from naturally-occurring subunits or their close homologs. The term may also refer to moieties that function similarly to oligonucleotides, but have non-naturally-occurring portions. Thus, oligonucleotides may have altered sugar moieties or inter-sugar linkages. Exemplary among these are phosphorothioate and other sulfur containing species which are known in the art. In preferred embodiments, at least one of the phosphodiester bonds of the oligonucleotide has been substituted with a structure that functions to enhance the ability of the compositions to penetrate into the region of cells where the RNA whose activity is to be modulated is located. It is preferred that such substitutions comprise phosphorothioate bonds, methyl phosphonate bonds, or short chain alkyl or cycloalkyl structures. In accordance with other preferred embodiments, the phosphodiester bonds are substituted with structures which are, at once, substantially non-ionic and non-chiral, or with structures which are chiral and enantiomerically specific. Persons of ordinary skill in the art will be able to select other linkages for use in the practice of the invention. Oligonucleotides may also include species that include at least some modified base forms. Thus, purines and pyrimidines other than those normally found in nature may be so employed. Similarly, modifications on the furanosyl portions of the nucleotide subunits may also be effected, as long as the essential tenets of this invention are adhered to. Examples of such modifications are 2′-O-alkyl- and 2′-halogen-substituted nucleotides. Some non-limiting examples of modifications at the 2′ position of sugar moieties which are useful in the present invention include OH, SH, SCH3, F, OCH3, OCN, O(CH2), NH2 and O(CH2)n CH3, where n is from 1 to about 10. Such oligonucleotides are functionally interchangeable with natural oligonucleotides or synthesized oligonucleotides, which have one or more differences from the natural structure. All such analogs are comprehended by this invention so long as they function effectively to hybridize with at least one gene from Tables 2-4 DNA or RNA to inhibit the function thereof.

The oligonucleotides in accordance with this invention preferably comprise from about 3 to about 50 subunits. It is more preferred that such oligonucleotides and analogs comprise from about 8 to about 25 subunits and still more preferred to have from about 12 to about 20 subunits. As defined herein, a “subunit” is a base and sugar combination suitably bound to adjacent subunits through phosphodiester or other bonds.

Antisense nucleic acids or oligonulcleotides can be produced by standard techniques (see, e.g., Shewmaker et al., U.S. Pat. No. 6,107,065). The oligonucleotides used in accordance with this invention may be conveniently and routinely made through the well-known technique of solid phase synthesis. Any other means for such synthesis may also be employed; however, the actual synthesis of the oligonucleotides is well within the abilities of the practitioner. It is also well known to prepare other oligonucleotides such as phosphorothioates and alkylated derivatives.

The oligonucleotides of this invention are designed to be hybridizable with RNA (e.g., mRNA) or DNA from genes described in Tables 2-4. For example, an oligonucleotide (e.g., DNA oligonucleotide) that hybridizes to mRNA from a gene described in Tables 2-4 can be used to target the mRNA for RnaseH digestion. Alternatively an oligonucleotide that can hybridize to the translation initiation site of the mRNA of a gene described in Tables 2-4 can be used to prevent translation of the mRNA. In another approach, oligonucleotides that bind to the double-stranded DNA of a gene from Tables 2-4 can be administered. Such oligonucleotides can form a triplex construct and inhibit the transcription of the DNA encoding polypeptides of the genes described in Tables 2-4. Triple helix pairing prevents the double helix from opening sufficiently to allow the binding of polymerases, transcription factors, or regulatory molecules. Recent therapeutic advances using triplex DNA have been described (see, e.g., J. E. Gee et al., 1994, Molecular and Immunologic Approaches, Futura Publishing Co., Mt. Kisco, N.Y.).

As non-limiting examples, antisense oligonucleotides may be targeted to hybridize to the following regions: mRNA cap region; translation initiation site; translational termination site; transcription initiation site; transcription termination site; polyadenylation signal; 3′ untranslated region; 5′ untranslated region; 5′ coding region; mid coding region; 3′ coding region; DNA replication initiation and elondation sites. Preferably, the complementary oligonucleotide is designed to hybridize to the most unique 5′ sequence of a gene described in Tables 2-4, including any of about 15-35 nucleotides spanning the 5′ coding sequence. In accordance with the present invention, the antisense oligonucleotide can be synthesized, formulated as a pharmaceutical composition, and administered to a subject. The synthesis and utilization of antisense and triplex oligonucleotides have been previously described (e.g., Simon et al., 1999; Barre et al., 2000; Elez et al., 2000; Sauter et al., 2000).

Alternatively, expression vectors derived from retroviruses, adenovirus, herpes or vaccinia viruses or from various bacterial plasmids may be used for delivery of nucleotide sequences to the targeted organ, tissue or cell population. Methods which are well known to those skilled in the art can be used to construct recombinant vectors which will express nucleic acid sequence that is complementary to the nucleic acid sequence encoding a polypeptide from the genes described in Tables 2-4. These techniques are described both in Sambrook et al., 1989 and in Ausubel et al., 1992. For example, expression of at least one gene from Tables 2-4 can be inhibited by transforming a cell or tissue with an expression vector that expresses high levels of untranslatable sense or antisense sequences. Even in the absence of integration into the DNA, such vectors may continue to transcribe RNA molecules until they are disabled by endogenous nucleases. Transient expression may last for a month or more with a nonreplicating vector, and even longer if appropriate replication elements are included in the vector system. Various assays may be used to test the ability of gene-specific antisense oligonucleotides to inhibit the expression of at least one gene from Tables 2-4. For example, mRNA levels of the genes described in Tables 2-4 can be assessed by Northern blot analysis (Sambrook et al., 1989; Ausubel et al., 1992; J. C. Alwine et al. 1977; I. M. Bird, 1998), quantitative or semi-quantitative RT-PCR analysis (see, e.g., W. M. Freeman et al., 1999; Ren et al., 1998; J. M. Cale et al., 1998), or in situ hybridization (reviewed by A. K. Raap, 1998). Alternatively, antisense oligonucleotides may be assessed by measuring levels of the polypeptide from the genes described in Tables 2-4, e.g., by western blot analysis, indirect immunofluorescence and immunoprecipitation techniques (see, e.g., J. M. Walker, 1998, Protein Protocols on cD-ROM, Humana Press, Totowa, N.J.). Any other means for such detection may also be employed, and is well within the abilities of the practitioner.

### Mapping Technologies

The present invention includes various methods which employ mapping technologies to map SNPs and polymorphisms. For purpose of clarity, this section comprises, but is not limited to, the description of mapping technologies that can be utilized to achieve the embodiments described herein. Mapping technologies may be based on amplification methods, restriction enzyme cleavage methods, hybridization methods, sequencing methods, and cleavage methods using agents.

Amplification methods include: self sustained sequence replication (Guatelli et al., 1990), transcriptional amplification system (Kwoh et al., 1989), Q-Beta Replicase (Lizardi et al., 1988), isothermal amplification (e.g. Dean et al., 2002; and Hafner et al., 2001), or any other nucleic acid amplification method, followed by the detection of the amplified molecules using techniques well known to those of ordinary skill in the art. These detection schemes are especially useful for the detection of nucleic acid molecules if such molecules are present in very low number.

Restriction enzyme cleavage methods include: isolating sample and control DNA, amplification (optional), digestion with one or more restriction endonucleases, determination of fragment length sizes by gel electrophoresis and comparing samples and controls. Differences in fragment length sizes between sample and control DNA indicates mutations in the sample DNA. Moreover, sequence specific ribozymes (see, e.g., U.S. Pat. No. 5,498,531 or DNAzyme e.g. U.S. Pat. No. 5,807,718) can be used to score for the presence of specific mutations by development or loss of a ribozyme or DNAzyme cleavage site.

Hybridization methods include any measurement of the hybridization or gene expression levels, of sample nucleic acids to probes corresponding to about 2, 3, 4, 5, 6, 7, 8, 9, 10, 15, 20, 25, 30, 50, 75, 100, 200, 500, 1000 or more genes, or ranges of these numbers, such as about 5-20, about 10-20, about 20-50, about 50-100, or about 100-200 genes of Tables 2-4.

SNPs and SNP maps of the invention can be identified or generated by hybridizing sample nucleic acids, e.g., DNA or RNA, to high density arrays or bead arrays containing oligonucleotide probes corresponding to the polymorphisms of Tables 5.1, 5.2, 6.2 and 6.3 (see the Affymetrix arrays and Illumina bead sets at www.affymetrix.com and www.illumina.com and see Cronin et al., 1996; or Kozal et al., 1996).

Methods of forming high density arrays of oligonucleotides with a minimal number of synthetic steps are known. The oligonucleotide analogue array can be synthesized on a single or on multiple solid substrates by a variety of methods, including, but not limited to, light-directed chemical coupling, and mechanically directed coupling (see Pirrung, U.S. Pat. No. 5,143,854).

In brief, the light-directed combinatorial synthesis of oligonucleotide arrays on a glass surface precedes using automated phosphoramidite chemistry and chip masking techniques. In one specific implementation, a glass surface is derivatized with a silane reagent containing a functional group, e.g., a hydroxyl or amine group blocked by a photolabile protecting group. Photolysis through a photolithogaphic mask is used selectively to expose functional groups which are then ready to react with incoming 5′ photoprotected nucleoside phosphoramidites. The phosphoramidites react only with those sites which are illuminated (and thus exposed by removal of the photolabile blocking group). Thus, the phosphoramidites only add to those areas selectively exposed from the preceding step. These steps are repeated until the desired array of sequences have been synthesized on the solid surface. Combinatorial synthesis of different oligonucleotide analogues at different locations on the array is determined by the pattern of illumination during synthesis and the order of addition of coupling reagents.

In addition to the foregoing, additional methods which can be used to generate an array of oligonucleotides on a single substrate are described in PCT Publication Nos. WO 93/09668 and WO 01/23614. High density nucleic acid arrays can also be fabricated by depositing pre-made or natural nucleic acids in predetermined positions. Synthesized or natural nucleic acids are deposited on specific locations of a substrate by light directed targeting and oligonucleotide directed targeting. Another embodiment uses a dispenser that moves from region to region to deposit nucleic acids in specific spots.

Nucleic acid hybridization simply involves contacting a probe and target nucleic acid under conditions where the probe and its complementary target can form stable hybrid duplexes through complementary base pairing. See WO 99/32660. The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids. Under low stringency conditions (e.g., low temperature and/or high salt) hybrid duplexes (e.g., DNA:DNA, RNA:RNA, or RNA:DNA) will form even where the annealed sequences are not perfectly complementary. Thus, specificity of hybridization is reduced at lower stringency. Conversely, at higher stringency (e.g., higher temperature or lower salt) successful hybridization tolerates fewer mismatches. One of skill in the art will appreciate that hybridization conditions may be selected to provide any degree of stringency.

In a preferred embodiment, hybridization is performed at low stringency to ensure hybridization and then subsequent washes are performed at higher stringency to eliminate mismatched hybrid duplexes. Successive washes may be performed at increasingly higher stringency until a desired level of hybridization specificity is obtained. Stringency can also be increased by addition of agents such as formamide. Hybridization specificity may be evaluated by comparison of hybridization to the test probes with hybridization to the various controls that can be present (e.g., expression level control, normalization control, mismatch controls, etc.).

In general, there is a tradeoff between hybridization specificity (stringency) and signal intensity. Thus, in a preferred embodiment, the wash is performed at the highest stringency that produces consistent results and that provides a signal intensity greater than approximately 10% of the background intensity. Thus, in a preferred embodiment, the hybridized array may be washed at successively higher stringency solutions and read between each wash. Analysis of the data sets thus produced will reveal a wash stringency above which the hybridization pattern is not appreciably altered and which provides adequate signal for the particular oligonucleotide probes of interest.

Probes based on the sequences of the genes described above may be prepared by any commonly available method. Oligonucleotide probes for screening or assaying a tissue or cell sample are preferably of sufficient length to specifically hybridize only to appropriate, complementary genes or transcripts. Typically the oligonucleotide probes will be at least about 10, 12, 14, 16, 18, 20 or 25 nucleotides in length. In some cases, longer probes of at least 30, 40, or 50 nucleotides will be desirable.

As used herein, oligonucleotide sequences that are complementary to one or more of the genes or gene fragments described in Tables 2-4 refer to oligonucleotides that are capable of hybridizing under stringent conditions to at least part of the nucleotide sequences of said genes. Such hybridizable oligonucleotides will typically exhibit at least about 75% sequence identity at the nucleotide level to said genes, preferably about 80% or 85% sequence identity or more preferably about 90% or 95% or more sequence identity to said genes (see GeneChip® Expression Analysis Manual, Affymetrix, Rev. 3, which is herein incorporated by reference in its entirety).

The phrase “hybridizing specifically to” or “specifically hybridizes” refers to the binding, duplexing, or hybridizing of a molecule substantially to or only to a particular nucleotide sequence or sequences under stringent conditions when that sequence is present in a complex mixture (e.g., total cellular) DNA or RNA.

As used herein a “probe” is defined as a nucleic acid, capable of binding to a target nucleic acid of complementary sequence through one or more types of chemical bonds, usually through complementary base pairing, usually through hydrogen bond formation. As used herein, a probe may include natural (i.e., A, G, U, C, or T) or modified bases (7-deazaguanosine, inosine, etc.). In addition, the bases in probes may be joined by a linkage other than a phosphodiester bond, so long as it does not interfere with hybridization. Thus, probes may be peptide nucleic acids in which the constituent bases are joined by peptide bonds rather than phosphodiester linkages.

A variety of sequencing reactions known in the art can be used to directly sequence nucleic acids for the presence or the absence of one or more polymorphisms of Tables 5.1, 5.2, 6.2 and 6.3. Examples of sequencing reactions include those based on techniques developed by Maxam and Gilbert (1977) or Sanger (1977). It is also contemplated that any of a variety of automated sequencing procedures can be utilized, including sequencing by mass spectrometry (see, e.g. PCT International Publication No. WO 94/16101; Cohen et al., 1996; and Griffin et al., 1993), real-time pyrophosphate sequencing method (Ronaghi et al., 1998; and Permutt et al., 2001) and sequencing by hybridization (see e.g. Drmanac et al., 2002).

Other methods of detecting polymorphisms include methods in which protection from cleavage agents is used to detect mismatched bases in RNA/RNA, DNA/DNA or RNA/DNA heteroduplexes (Myers et al., 1985). In general, the technique of “mismatch cleavage” starts by providing heteroduplexes formed by hybridizing (labeled) RNA or DNA containing a wild-type sequence with potentially mutant RNA or DNA obtained from a sample. The double-stranded duplexes are treated with an agent who cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of a mutation or SNP (see, for example, Cotton et al., 1988; and Saleeba et al., 1992). In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping polymorphisms. For example, the mutY enzyme of E. coli cleaves A at G/A mismatches (Hsu et al., 1994). Other examples include, but are not limited to, the MutHLS enzyme complex of E. coli (Smith and Modrich Proc. 1996) and Cel 1 from the celery (Kulinski et al., 2000) both cleave the DNA at various mismatches. According to an exemplary embodiment, a probe based on a polymorphic site corresponding to a polymorphism of Tables 5.1, 5.2, 6.2 and 6.3 is hybridized to a cDNA or other DNA product from a test cell or cells. The duplex is treated with a DNA mismatch repair enzyme, and the cleavage products, if any, can be detected from electrophoresis protocols or the like. See, for example, U.S. Pat. No. 5,459,039. Alternatively, the screen can be performed in vivo following the insertion of the heteroduplexes in an appropriate vector. The whole procedure is known to those ordinary skilled in the art and is referred to as mismatch repair detection (see e.g. Fakhrai-Rad et al., 2004).

In other embodiments, alterations in electrophoretic mobility can be used to identify polymorphisms in a sample. For example, single strand conformation polymorphism (SSCP) analysis can be used to detect differences in electrophoretic mobility between mutant and wild type nucleic acids (Orita et al., 1989; Cotton et al., 1993; and Hayashi 1992). Single-stranded DNA fragments of case and control nucleic acids will be denatured and allowed to renature. The secondary structure of single-stranded nucleic acids varies according to sequence. The resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the method utilizes heteroduplex analysis to separate double stranded heteroduplex molecules on the basis of changes in electrophoretic mobility (Kee et al., 1991).

In yet another embodiment, the movement of mutant or wild-type fragments in a polyacrylamide gel containing a gradient of denaturant is assayed using denaturing gradient gel electrophoresis (DGGE) (Myers et al., 1985). When DGGE is used as the method of analysis, DNA will be modified to insure that it does not completely denature, for example by adding a GC clamp of approximately 40 by of high-melting GC-rich DNA by PCR. In a further embodiment, a temperature gradient is used in place of a denaturing gradient to identify differences in the mobility of control and sample DNA (Rosenbaum et al., 1987). In another embodiment, the mutant fragment is detected using denaturing HPLC (see e.g. Hoogendoorn et al., 2000).

Examples of other techniques for detecting polymorphisms include, but are not limited to, selective oligonucleotide hybridization, selective amplification, selective primer extension, selective ligation, single-base extension, selective termination of extension or invasive cleavage assay. For example, oligonucleotide primers may be prepared in which the polymorphism is placed centrally and then hybridized to target DNA under conditions which permit hybridization only if a perfect match is found (Saiki et al., 1986; Saiki et al., 1989). Such oligonucleotides are hybridized to PCR amplified target DNA or a number of different mutations when the oligonucleotides are attached to the hybridizing membrane and hybridized with labeled target DNA. Alternatively, the amplification, the allele-specific hybridization and the detection can be done in a single assay following the principle of the 5′ nuclease assay (e.g. see Livak et al., 1995). For example, the associated allele, a particular allele of a polymorphic locus, or the like is amplified by PCR in the presence of both allele-specific oligonucleotides, each specific for one or the other allele. Each probe has a different fluorescent dye at the 5′ end and a quencher at the 3′ end. During PCR, if one or the other or both allele-specific oligonucleotides are hybridized to the template, the Taq polymerase via its 5′ exonuclease activity will release the corresponding dyes. The latter will thus reveal the genotype of the amplified product.

Hybridization assays may also be carried out with a temperature gradient following the principle of dynamic allele-specific hybridization or like e.g. Jobs et al., (2003); and Bourgeois and Labuda, (2004). For example, the hybridization is done using one of the two allele-specific oligonucleotides labeled with a fluorescent dye, and an intercalating quencher under a gradually increasing temperature. At low temperature, the probe is hybridized to both the mismatched and full-matched template. The probe melts at a lower temperature when hybridized to the template with a mismatch. The release of the probe is captured by an emission of the fluorescent dye, away from the quencher. The probe melts at a higher temperature when hybridized to the template with no mismatch. The temperature-dependent fluorescence signals therefore indicate the absence or presence of an associated allele, a particular allele of a polymorphic locus, or the like (e.g. Jobs et al., 2003). Alternatively, the hybridization is done under a gradually decreasing temperature. In this case, both allele-specific oligonucleotides are hybridized to the template competitively. At high temperature none of the two probes are hybridized. Once the optimal temperature of the full-matched probe is reached, it hybridizes and leaves no target for the mismatched probe (e.g. Bourgeois and Labuda, 2004). In the latter case, if the allele-specific probes are differently labeled, then they are hybridized to a single PCR-amplified target. If the probes are labeled with the same dye, then the probe cocktail is hybridized twice to identical templates with only one labeled probe, different in the two cocktails, in the presence of the unlabeled competitive probe.

Alternatively, allele specific amplification technology that depends on selective PCR amplification may be used in conjunction with the present invention. Oligonucleotides used as primers for specific amplification may carry the associated allele, a particular allele of a polymorphic locus, or the like, also referred to as “mutation” of interest in the center of the molecule, so that amplification depends on differential hybridization (Gibbs et al., 1989) or at the extreme 3′ end of one primer where, under appropriate conditions, mismatch can prevent, or reduce polymerase extension (Prossner, 1993). In addition it may be desirable to introduce a novel restriction site in the region of the mutation to create cleavage-based detection (Gasparini et al., 1992). It is anticipated that in certain embodiments, amplification may also be performed using Taq ligase for amplification (Barany, 1991). In such cases, ligation will occur only if there is a perfect match at the 3′ end of the 5′ sequence making it possible to detect the presence of a known associated allele, a particular allele of a polymorphic locus, or the like at a specific site by looking for the presence or absence of amplification. The products of such an oligonucleotide ligation assay can also be detected by means of gel electrophoresis. Furthermore, the oligonucleotides may contain universal tags used in PCR amplification and zip code tags that are different for each allele. The zip code tags are used to isolate a specific, labeled oligonucleotide that may contain a mobility modifier (e.g. Grossman et al., 1994).

In yet another alternative, allele-specific elongation followed by ligation will form a template for PCR amplification. In such cases, elongation will occur only if there is a perfect match at the 3′ end of the allele-specific oligonucleotide using a DNA polymerase. This reaction is performed directly on the genomic DNA and the extension/ligation products are amplified by PCR. To this end, the oligonucleotides contain universal tags allowing amplification at a high multiplex level and a zip code for SNP identification. The PCR tags are designed in such a way that the two alleles of a SNP are amplified by different forward primers, each having a different dye. The zip code tags are the same for both alleles of a given SNPs and they are used for hybridization of the PCR-amplified products to oligonucleotides bound to a solid support, chip, bead array or like. For an example of the procedure, see Fan et al. (Cold Spring Harbor Symposia on Quantitative Biology, Vol. LXVIII, pp. 69-78 2003).

Another alternative includes the single-base extension/ligation assay using a molecular inversion probe, consisting of a single, long oligonucleotide (see e.g. Hardenbol et al., 2003). In such an embodiment, the oligonucleotide hybridizes on both side of the SNP locus directly on the genomic DNA, leaving a one-base gap at the SNP locus. The gap-filling, one-base extension/ligation is performed in four tubes, each having a different dNTP. Following this reaction, the oligonucleotide is circularized whereas unreactive, linear oligonucleotides are degraded using an exonuclease such as exonuclease I of E. coli. The circular oligonucleotides are then linearized and the products are amplified and labeled using universal tags on the oligonucleotides. The original oligonucleotide also contains a SNP-specific zip code allowing hybridization to oligonucleotides bound to a solid support, chip, and bead array or like. This reaction can be performed at a high multiplexed level.

In another alternative, the associated allele, a particular allele of a polymorphic locus, or the like is scored by single-base extension (see e.g. U.S. Pat. No. 5,888,819). The template is first amplified by PCR. The extension oligonucleotide is then hybridized next to the SNP locus and the extension reaction is performed using a thermostable polymerase such as ThermoSequenase (GE Healthcare) in the presence of labeled ddNTPs. This reaction can therefore be cycled several times. The identity of the labeled ddNTP incorporated will reveal the genotype at the SNP locus. The labeled products can be detected by means of gel electrophoresis, fluorescence polarization (e.g. Chen et al., 1999) or by hybridization to oligonucleotides bound to a solid support, chip, and bead array or like. In the latter case, the extension oligonucleotide will contain a SNP-specific zip code tag.

In yet another alternative, a SNP is scored by selective termination of extension. The template is first amplified by PCR and the extension oligonucleotide hybridizes in the vicinity of the SNP locus, close to but not necessarily adjacent to it. The extension reaction is carried out using a thermostable polymerase such as ThermoSequenase (GE Healthcare) in the presence of a mix of dNTPs and at least one ddNTP. The latter has to terminate the extension at one of the allele of the interrogated SNP, but not both such that the two alleles will generate extension products of different sizes. The extension product can then be detected by means of gel electrophoresis, in which case the extension products need to be labeled, or by mass spectrometry (see e.g. Storm et al., 2003).

In another alternative, SNPs are detected using an invasive cleavage assay (see U.S. Pat. No. 6,090,543). There are five oligonucleotides per SNP to interrogate but these are used in a two step-reaction. During the primary reaction, three of the designed oligonucleotides are first hybridized directly to the genomic DNA. One of them is locus-specific and hybridizes up to the SNP locus (the pairing of the 3′ base at the SNP locus is not necessary). There are two allele-specific oligonucleotides that hybridize in tandem to the locus-specific probe but also contain a 5′ flap that is specific for each allele of the SNP. Depending upon hybridization of the allele-specific oligonucleotides at the base of the SNP locus, this creates a structure that is recognized by a cleavase enzyme (U.S. Pat. No. 6,090,606) and the allele-specific flap is released. During the secondary reaction, the flap fragments hybridize to a specific cassette to recreate the same structure as above except that the cleavage will release a small DNA fragment labeled with a fluorescent dye that can be detected using regular fluorescence detector. In the cassette, the emission of the dye is inhibited by a quencher.

Methods to Identify Agents that Modulate the Expression of a Nucleic Acid Encoding a Gene Involved in IBD

The present invention provides methods for identifying agents that modulate the expression of a nucleic acid encoding a gene from Tables 2-4. Such methods may utilize any available means of monitoring for changes in the expression level of the nucleic acids of the invention. As used herein, an agent is said to modulate the expression of a nucleic acid of the invention if it is capable of up- or down-regulating expression of the nucleic acid in a cell. Such cells can be obtained from any parts of the body such as the hair, mouth, rectum, scalp, blood, dermis, epidermis, skin cells, cutaneous surfaces, intertrigious areas, genitalia and fluids, vessels and endothelium. Some non-limiting examples of cells that can be used are: digestive system cells, muscle cells, nervous cells, blood and vessels cells, T cell, mast cell, lymphocyte, monocyte, macrophage, and epithelial cells.

In one assay format, the expression of a nucleic acid encoding a gene of the invention (see Tables 2-4) in a cell or tissue sample is monitored directly by hybridization to the nucleic acids of the invention. Cell lines or tissues are exposed to the agent to be tested under appropriate conditions and time and total RNA or mRNA is isolated by standard procedures such as those disclosed in Sambrook et al., (1989) Molecular Cloning: A Laboratory Manual, Cold Spring Harbor Laboratory Press).

Probes to detect differences in RNA expression levels between cells exposed to the agent and control cells may be prepared as described above. Hybridization conditions are modified using known methods, such as those described by Sambrook at al., and Ausubel et al., as required for each probe. Hybridization of total cellular RNA or RNA enriched for polyA RNA can be accomplished in any available format. For instance, total cellular RNA or RNA enriched for polyA RNA can be affixed to a solid support and the solid support exposed to at least one probe comprising at least one, or part of one of the sequences of the invention under conditions in which the probe will specifically hybridize. Alternatively, nucleic acid fragments comprising at least one, or part of one of the sequences of the invention can be affixed to a solid support, such as a silicon chip or a porous glass wafer. The chip or wafer can then be exposed to total cellular RNA or polyA RNA from a sample under conditions in which the affixed sequences will specifically hybridize to the RNA. By examining for the ability of a given probe to specifically hybridize to an RNA sample from an untreated cell population and from a cell population exposed to the agent, agents which up or down regulate expression are identified.

Methods to Identify Agents that Modulate the Activity of a Protein Encoded by a Gene Involved in IBD

The present invention provides methods for identifying agents that modulate at least one activity of the proteins described in Tables 2-4. Such methods may utilize any means of monitoring or detecting the desired activity. As used herein, an agent is said to modulate the expression of a protein of the invention if it is capable of up- or down-regulating expression of the protein in a cell. Such cells can be obtained from any parts of the body such as the hair, mouth, rectum, scalp, blood, dermis, epidermis, skin cells, cutaneous surfaces, intertrigious areas, genitalia and fluids, vessels and endothelium. Some non-limiting examples of cells that can be used are: digestive system cells, muscle cells, nervous cells, blood and vessels cells, T cell, mast cell, lymphocyte, monocyte, macrophage, and epithelial cells.

In one format, the specific activity of a protein of the invention, normalized to a standard unit, may be assayed in a cell population that has been exposed to the agent to be tested and compared to an unexposed control cell population. Cell lines or populations are exposed to the agent to be tested under appropriate conditions and times. Cellular lysates may be prepared from the exposed cell line or population and a control, unexposed cell line or population. The cellular lysates are then analyzed with a probe, such as an antibody probe.

Antibody probes can be prepared by immunizing suitable mammalian hosts utilizing appropriate immunization protocols using the proteins of the invention or antigen-containing fragments thereof. To enhance immunogenicity, these proteins or fragments can be conjugated to suitable carriers. Methods for preparing immunogenic conjugates with carriers such as BSA, KLH or other carrier proteins are well known in the art. In some circumstances, direct conjugation using, for example, carbodiimide reagents may be effective; in other instances linking reagents such as those supplied by Pierce Chemical Co. (Rockford, Ill.) may be desirable to provide accessibility to the hapten. The hapten peptides can be extended at either the amino or carboxy terminus with a cysteine residue or interspersed with cysteine residues, for example, to facilitate linking to a carrier. Administration of the immunogens is conducted generally by injection over a suitable time period and with use of suitable adjuvants, as is generally understood in the art. During the immunization schedule, titers of antibodies are taken to determine adequacy of antibody formation. While the polyclonal antisera produced in this way may be satisfactory for some applications, for pharmaceutical compositions, use of monoclonal preparations is preferred. Immortalized cell lines which secrete the desired monoclonal antibodies may be prepared using standard methods, see e.g., Kohler & Milstein (1992) or modifications which affect immortalization of lymphocytes or spleen cells, as is generally known. The immortalized cell lines secreting the desired antibodies can be screened by immunoassay in which the antigen is the peptide hapten, polypeptide or protein. When the appropriate immortalized cell culture secreting the desired antibody is identified, the cells can be cultured either in vitro or by production in ascites fluid. The desired monoclonal antibodies may be recovered from the culture supernatant or from the ascites supernatant. Fragments of the monoclonal antibodies or the polyclonal antisera which contain the immunologically significant portion(s) can be used as antagonists, as well as the intact antibodies. Use of immunologically reactive fragments, such as Fab or Fab′ fragments, is often preferable, especially in a therapeutic context, as these fragments are generally less immunogenic than the whole immunoglobulin. The antibodies or fragments may also be produced, using current technology, by recombinant means. Antibody regions that bind specifically to the desired regions of the protein can also be produced in the context of chimeras derived from multiple species. Antibody regions that bind specifically to the desired regions of the protein can also be produced in the context of chimeras from multiple species, for instance, humanized antibodies. The antibody can therefore be a humanized antibody or a human antibody, as described in U.S. Pat. No. 5,585,089 or Riechmann et al. (1988).

Agents that are assayed in the above method can be randomly selected or rationally selected or designed. As used herein, an agent is said to be randomly selected when the agent is chosen randomly without considering the specific sequences involved in the association of the protein of the invention alone or with its associated substrates, binding partners, etc. An example of randomly selected agents is the use of a chemical library or a peptide combinatorial library, or a growth broth of an organism. As used herein, an agent is said to be rationally selected or designed when the agent is chosen on a non-random basis which takes into account the sequence of the target site or its conformation in connection with the agent's action. Agents can be rationally selected or rationally designed by utilizing the peptide sequences that make up these sites. For example, a rationally selected peptide agent can be a peptide whose amino acid sequence is identical to or a derivative of any functional consensus site. The agents of the present invention can be, as examples, oligonucleotides, antisense polynucleotides, interfering RNA, peptides, peptide mimetics, antibodies, antibody fragments, small molecules, vitamin derivatives, as well as carbohydrates. Peptide agents of the invention can be prepared using standard solid phase (or solution phase) peptide synthesis methods, as is known in the art. In addition, the DNA encoding these peptides may be synthesized using commercially available oligonucleotide synthesis instrumentation and produced recombinantly using standard recombinant production systems. The production using solid phase peptide synthesis is necessitated if non-gene-encoded amino acids are to be included.

Another class of agents of the present invention includes antibodies or fragments thereof that bind to a protein encoded by a gene in Tables 2-4. Antibody agents can be obtained by immunization of suitable mammalian subjects with peptides, containing as antigenic regions, those portions of the protein intended to be targeted by the antibodies (see section above of antibodies as probes for standard antibody preparation methodologies).

In yet another class of agents, the present invention includes peptide mimetics that mimic the three-dimensional structure of the protein encoded by a gene from Tables 2-4. Such peptide mimetics may have significant advantages over naturally occurring peptides, including, for example: more economical production, greater chemical stability, enhanced pharmacological properties (half-life, absorption, potency, efficacy, etc.), altered specificity (e.g., a broad-spectrum of biological activities), reduced antigenicity and others. In one form, mimetics are peptide-containing molecules that mimic elements of protein secondary structure. The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. In another form, peptide analogs are commonly used in the pharmaceutical industry as non-peptide drugs with properties analogous to those of the template peptide. These types of non-peptide compounds are also referred to as peptide mimetics or peptidomimetics (Fauchere, 1986; Veber & Freidinger, 1985; Evans et al., 1987) which are usually developed with the aid of computerized molecular modeling. Peptide mimetics that are structurally similar to therapeutically useful peptides may be used to produce an equivalent therapeutic or prophylactic effect. Generally, peptide mimetics are structurally similar to a paradigm polypeptide (i.e., a polypeptide that has a biochemical property or pharmacological activity), but have one or more peptide linkages optionally replaced by a linkage using methods known in the art. Labeling of peptide mimetics usually involves covalent attachment of one or more labels, directly or through a spacer (e.g., an amide group), to non-interfering position(s) on the peptide mimetic that are predicted by quantitative structure-activity data and molecular modeling. Such non-interfering positions generally are positions that do not form direct contacts with the macromolecule(s) to which the peptide mimetic binds to produce the therapeutic effect. Derivitization (e.g., labeling) of peptide mimetics should not substantially interfere with the desired biological or pharmacological activity of the peptide mimetic. The use of peptide mimetics can be enhanced through the use of combinatorial chemistry to create drug libraries. The design of peptide mimetics can be aided by identifying amino acid mutations that increase or decrease binding of the protein to its binding partners. Approaches that can be used include the yeast two hybrid method (see Chien et al., 1991) and the phage display method. The two hybrid method detects protein-protein interactions in yeast (Fields et al., 1989). The phage display method detects the interaction between an immobilized protein and a protein that is expressed on the surface of phages such as lambda and M13 (Amberg et al., 1993; Hogrefe et al., 1993). These methods allow positive and negative selection for protein-protein interactions and the identification of the sequences that determine these interactions.

Method to Diagnose 1BD (e.g. Crohn's Disease)

The present invention also relates to methods for diagnosing CROHN or a related disease, preferably a subtype of IBD (e.g. Crohn's disease), a predisposition to such a disease and/or disease progression. In some methods, the steps comprise contacting a target sample with (a) nucleic acid molecule(s) or fragments thereof and comparing the concentration of individual mRNA(s) with the concentration of the corresponding mRNA(s) from at least one healthy donor. An aberrant (increased or decreased) mRNA level of at least one gene from Tables 2-4, at least 5 or 10 genes from Tables 2-4, at least 50 genes from Tables 2-4, at least 100 genes from Tables 2-4 or at least 200 genes from Tables 2-4 determined in the sample in comparison to the control sample is an indication of IBD (e.g. Crohn's disease) a related subtype or a disposition to such kinds of diseases. For diagnosis, samples are, preferably, obtained from any parts of the body such as the hair, mouth, rectum, scalp, blood, dermis, epidermis, skin cells, cutaneous surfaces, intertrigious areas, genitalia and fluids, vessels and endothelium. Some non-limiting examples of cells that can be used are: cells of the digestive system, muscle cells, nervous cells, blood and vessels cells, T cell, mast cell, lymphocyte, monocyte, macrophage, and epithelial cells.

For analysis of gene expression, total RNA is obtained from cells according to standard procedures and, preferably, reverse-transcribed. Preferably, a DNAse treatment (in order to get rid of contaminating genomic DNA) is performed.

The nucleic acid molecule or fragment is typically a nucleic acid probe for hybridization or a primer for PCR. The person skilled in the art is in a position to design suitable nucleic acids probes based on the information provided in the Tables of the present invention (Tables 1-4, 5.1, 5.2, 6.2, 6.3). The target cellular component, i.e. mRNA, e.g., in brain tissue, may be detected directly in situ, e.g. by in situ hybridization or it may be isolated from other cell components by common methods known to those skilled in the art before contacting with a probe. Detection methods include Northern blot analysis, RNase protection, in situ methods, e.g. in situ hybridization, in vitro amplification methods (PCR, LCR, QRNA replicase or RNA-transcription/amplification (TAS, 3SR), reverse dot blot disclosed in EP-B10237362) and other detection assays that are known to those skilled in the art. Products obtained by in vitro amplification can be detected according to established methods, e.g. by separating the products on agarose or polyacrylamide gels and by subsequent staining with ethidium bromide or any other dye or reagent. Alternatively, the amplified products can be detected by using labeled primers for amplification or labeled dNTPs. Preferably, detection is based on a microarray.

The probes (or primers) (or, alternatively, the reverse-transcribed sample mRNAs) can be detectably labeled, for example, with a radioisotope, a bioluminescent compound, a chemiluminescent compound, a fluorescent compound, a metal chelate, or an enzyme.

The present invention also relates to the use of the nucleic acid molecules or fragments described above for the preparation of a diagnostic composition for the diagnosis of IBD (e.g. Crohn's disease) or a subtype or predisposition to such a disease.

The present invention also relates to the use of the nucleic acid molecules of the present invention for the isolation or development of a compound which is useful for therapy of IBD (e.g. Crohn's disease). For example, the nucleic acid molecules of the invention and the data obtained using said nucleic acid molecules for diagnosis of IBD (e.g. Crohn's disease) might allow for the identification of further genes which are specifically dysregulated, and thus may be considered as potential targets for therapeutic interventions. Furthermore, such diagnostic might also be used for selection of patients that might respond positively or negatively to a potential target for therapeutic interventions (as for the pharmacogenomics and personalized medicine concept well know in the art; see prognostic assays text below).

The invention further provides prognostic assays that can be used to identify subjects having or at risk of developing IBD (e.g. Crohn's disease). In such method, a test sample is obtained from a subject and the amount and/or concentration of the nucleic acid described in Tables 2-4 is determined; wherein the presence of an associated allele, a particular allele of a polymorphic locus, or the likes in the nucleic acids sequences of this invention (see SEQ ID from Tables 5.1, 5.2, 6.2 and 6.3) can be diagnostic for a subject having or at risk of developing IBD (e.g. Crohn's disease). As used herein, a “test sample” refers to a biological sample obtained from a subject of interest. For example, a test sample can be a biological fluid, a cell sample, or tissue. A biological fluid can be, but is not limited to saliva, serum, mucus, urine, stools, spermatozoids, vaginal secretions, lymph, amiotic liquid, pleural liquid and tears. Cells can be, but are not limited to: cells of the digestive system, hair cells, muscle cells, nervous cells, blood and vessels cells, dermis, epidermis and other skin cells, and various brain cells.

Furthermore, the prognostic assays described herein can be used to determine whether a subject can be administered an agent (e.g., an agonist, antagonist, peptidomimetic, polypeptide, nucleic acid such as antisense DNA or interfering RNA (RNAi), small molecule or other drug candidate) to treat IBD (e.g. Crohn's disease). Specifically, these assays can be used to predict whether an individual will have an efficacious response or will experience adverse events in response to such an agent. For example, such methods can be used to determine whether a subject can be effectively treated with an agent that modulates the expression and/or activity of a gene from Tables 2-4 or the nucleic acids described herein. In another example, an association study may be performed to identify polymorphisms from Tables 5.1, 5.2, 6.2 and 6.3 that are associated with a given response to the agent, e.g., an efficacious response or the likelihood of one or more adverse events. Thus, one embodiment of the present invention provides methods for determining whether a subject can be effectively treated with an agent for a disease associated with aberrant expression or activity of a gene from Tables 2-4 in which a test sample is obtained and nucleic acids or polypeptides from Tables 2-4 are detected (e.g., wherein the presence of a particular level of expression of a gene from Tables 2-4 or a particular allelic variant of such gene, such as polymorphisms from Tables 5.1, 5.2, 6.2 and 6.3 is diagnostic for a subject that can be administered an agent to treat a disorder such as IBD (e.g. Crohn's disease). In one embodiment, the method includes obtaining a sample from a subject suspected of having IBD (e.g. Crohn's disease) or an affected individual and exposing such sample to an agent. The expression and/or activity of the nucleic acids and/or genes of the invention are monitored before and after treatment with such agent to assess the effect of such agent. After analysis of the expression values, one skilled in the art can determine whether such agent can effectively treat such subject. In another embodiment, the method includes obtaining a sample from a subject having or susceptible to developing IBD (e.g. Crohn's disease) and determining the allelic constitution of polymorphisms from Tables 5.1, 5.2, 6.2 and 6.3 that are associated with a particular response to an agent. After analysis of the allelic constitution of the individual at the associated polymorphisms, one skilled in the art can determine whether such agent can effectively treat such subject.

The methods of the invention can also be used to detect genetic alterations in a gene from Tables 2-4, thereby determining if a subject with the lesioned gene is at risk for a disease associated with IBD (e.g. Crohn's disease). In preferred embodiments, the methods include detecting, in a sample of cells from the subject, the presence or absence of a genetic alteration characterized by at least one alteration linked to or affecting the integrity of a gene from Tables 2-4 encoding a polypeptide or the misexpression of such gene. For example, such genetic alterations can be detected by ascertaining the existence of at least one of: (1) a deletion of one or more nucleotides from a gene from Tables 2-4; (2) an addition of one or more nucleotides to a gene from Tables 2-4; (3) a substitution of one or more nucleotides of a gene from Tables 2-4; (4) a chromosomal rearrangement of a gene from Tables 2-4; (5) an alteration in the level of a messenger RNA transcript of a gene from Tables 2-4; (6) aberrant modification of a gene from Tables 2-4, such as of the methylation pattern of the genomic DNA, (7) the presence of a non-wild type splicing pattern of a messenger RNA transcript of a gene from Tables 2-4; (8) inappropriate post-translational modification of a polypeptide encoded by a gene from Tables 2-4; and (9) alternative promoter use. As described herein, there are a large number of assay techniques known in the art which can be used for detecting alterations in a gene from Tables 2-4. A preferred biological sample is a peripheral blood sample obtained by conventional means from a subject. Another preferred biological sample is a buccal swab. Other biological samples can be, but are not limited to blood, biopsy sample, urine, stools, hair, vaginal secretions, lymph, amiotic liquid, pleural liquid and tears.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or alternatively, in a ligation chain reaction (LCR) (see, e.g., Landegran et al., 1988; and Nakazawa et al., 1994), the latter of which can be particularly useful for detecting point mutations in a gene from Tables 2-4 (see Abavaya et al., 1995). This method can include the steps of collecting a sample of cells from a patient, isolating nucleic acid (e.g., genomic DNA, mRNA, or both) from the cells of the sample, contacting the nucleic acid sample with one or more primers which specifically hybridize to a gene from Tables 2-4 under conditions such that hybridization and amplification of the nucleic acid from Tables 2-4 (if present) occurs, and detecting the presence or absence of an amplification product, or detecting the size of the amplification product and comparing the length to a control sample. PCR and/or LCR may be desirable to use as a preliminary amplification step in conjunction with some of the techniques used for detecting a mutation, an associated allele, a particular allele of a polymorphic locus, or the like described in the above sections. Other mutation detection and mapping methods are described in previous sections of the detailed description of the present invention.

The present invention also relates to further methods for diagnosing IBD (e.g. Crohn's disease) or a related disorder or subtype, a predisposition to such a disorder and/or disorder progression. In some methods, the steps comprise contacting a target sample with (a) nucleic molecule(s) or fragments thereof and determining the presence or absence of a particular allele of a polymorphism that confers a disorder-related phenotype (e.g., predisposition to such a disorder and/or disorder progression). The presence of at least one allele from Tables 5.1, 5.2, 6.2 and 6.3 that is associated with IBD (e.g. Crohn's disease) (“associated allele”), at least 5 or 10 associated alleles from Tables 5.1, 5.2, 6.2 and 6.3, at least 50 associated alleles from Tables 5.1, 5.2, 6.2 and 6.3 at least 100 associated alleles from Tables 5.1, 5.2, 6.2 and 6.3, or at least 200 associated alleles from Tables 5.1, 5.2, 6.2 and 6.3 determined in the sample is an indication of IBD (e.g. Crohn's disease) or a related disorder, a disposition or predisposition to such kinds of disorders, or a prognosis for such disorder progression. Such samples and cells can be obtained from any parts of the body such as the hair, colon, mouth, rectum, scalp, blood, dermis, epidermis, skin cells, cutaneous surfaces, intertrigious areas, genitalia and fluids, vessels and endothelium. Some non-limiting examples of cells that can be used are: cells of the digestive system, muscle cells, nervous cells, blood and vessels cells, T cell, mast cell, lymphocyte, monocyte, macrophage, and epithelial cells.

In other embodiments, alterations in a gene from Tables 2-4 can be identified by hybridizing sample and control nucleic acids, e.g., DNA or RNA, to high density arrays or bead arrays containing tens to thousands of oligonucleotide probes (Cronin et al., 1996; Kozal et al., 1996). For example, alterations in a gene from Tables 2-4 can be identified in two dimensional arrays containing light-generated DNA probes as described in Cronin et al., (1996). Briefly, a first hybridization array of probes can be used to scan through long stretches of DNA in a sample and control to identify base changes between the sequences by making linear arrays of sequential overlapping probes. This step allows the identification of point mutations, associated alleles, particular alleles of a polymorphic locus, or the like. This step is followed by a second hybridization array that allows the characterization of specific mutations by using smaller, specialized probe arrays complementary to all variants, mutations, alleles detected. Each mutation array is composed of parallel probe sets, one complementary to the wild-type gene and the other complementary to the mutant gene.

In yet another embodiment, any of a variety of sequencing reactions known in the art can be used to directly sequence a gene from Tables 2-4 and detect an associated allele, a particular allele of a polymorphic locus, or the like by comparing the sequence of the sample gene from Tables 2-4 with the corresponding wild-type (control) sequence (see text described in previous sections for various sequencing techniques and other methods of detecting an associated allele, a particular allele of a polymorphic locus, or the likes in a gene from Tables 2-4. Such methods include methods in which protection from cleavage agents is used to detect mismatched bases in RNA/RNA, DNA/DNA or RNA/DNA heteroduplexes (Myers et al., 1985) and alterations in electrophoretic mobility. Examples of other techniques for detecting point mutations, an associated allele, a particular allele of a polymorphic locus, or the like include, but are not limited to, selective oligonucleotide hybridization, selective amplification, selective primer extension, selective ligation, single-base extension, selective termination of extension or invasive cleavage assay.

Other types of markers can also be used for diagnostic purposes. For example, microsatellites can also be useful to detect the genetic predisposition of an individual to a given disorder. Microsatellites consist of short sequence motifs of one or a few nucleotides repeated in tandem. The most common motifs are polynucleotide runs, dinucleotide repeats (particularly the CA repeats) and trinucleotide repeats. However, other types of repeats can also be used. The microsatellites are very useful for genetic mapping because they are highly polymorphic in their length. Microsatellite markers can be typed by various means, including but not limited to DNA fragment sizing, oligonucleotide ligation assay and mass spectrometry. For example, the locus of the microsatellite is amplified by PCR and the size of the PCR fragment will be directly correlated to the length of the microsatellite repeat. The size of the PCR fragment can be detected by regular means of gel electrophoresis. The fragment can be labeled internally during PCR or by using end-labeled oligonucleotides in the PCR reaction (e.g. Mansfield et al., 1996). Alternatively, the size of the PCR fragment is determined by mass spectrometry. In another alternative, an oligonucleotide ligation assay can be performed. The microsatellite locus is first amplified by PCR. Then, different oligonucleotides can be submitted to ligation at the center of the repeat with a set of oligonucleotides covering all the possible lengths of the marker at a given locus (Zirvi et al., 1999). Another example of design of an oligonucleotide assay comprises the ligation of three oligonucleotides; a 5′ oligonucleotide hybridizing to the 5′ flanking sequence, a repeat oligonucleotide of the length of the shortest allele of the marker hybridizing to the repeated region and a set of 3′ oligonucleotides covering all the existing alleles hybridizing to the 3′ flanking sequence and a portion of the repeated region for all the alleles longer than the shortest one. For the shortest allele, the 3′ oligonucleotide exclusively hybridizes to the 3′ flanking sequence (U.S. Pat. No. 6,479,244).

The methods described herein may be performed, for example, by utilizing pre-packaged diagnostic kits comprising at least one probe nucleic acid selected from the SEQ ID of Tables 5.1, 5.2, 6.2 and 6.3, or antibody reagent described herein, which may be conveniently used, for example, in a clinical setting to diagnose patient exhibiting symptoms or a family history of a disorder or disorder involving abnormal activity of genes from Tables 2-4.

Method to Treat an Animal Suspected of Having IBD (e.g. Crohn's Disease)

The present invention provides methods of treating a disease associated with IBD (e.g. Crohn's disease) by expressing in vivo the nucleic acids of at least one gene from Tables 2-4. These nucleic acids can be inserted into any of a number of well-known vectors for the transfection of target cells and organisms as described below. The nucleic acids are transfected into cells, ex vivo or in vivo, through the interaction of the vector and the target cell. The nucleic acids encoding a gene from Tables 2-4, under the control of a promoter, then express the encoded protein, thereby mitigating the effects of absent, partial inactivation, or abnormal expression of a gene from Tables 2-4.

Such gene therapy procedures have been used to correct acquired and inherited genetic defects, cancer, and viral infection in a number of contexts. The ability to express artificial genes in humans facilitates the prevention and/or cure of many important human disorders, including many disorders which are not amenable to treatment by other therapies (for a review of gene therapy procedures, see Anderson, 1992; Nabel & Feigner, 1993; Mitani & Caskey, 1993; Mulligan, 1993; Dillon, 1993; Miller, 1992; Van Brunt, 1998; Vigne, 1995; Kremer & Perricaudet 1995; Doerfler & Bohm 1995; and Yu et al., 1994).

Delivery of the gene or genetic material into the cell is the first critical step in gene therapy treatment of a disorder. A large number of delivery methods are well known to those of skill in the art. Preferably, the nucleic acids are administered for in vivo or ex vivo gene therapy uses. Non-viral vector delivery systems include DNA plasmids, naked nucleic acid, and nucleic acid complexed with a delivery vehicle such as a liposome. Viral vector delivery systems include DNA and RNA viruses, which have either episomal or integrated genomes after delivery to the cell. For a review of gene therapy procedures, see the references included in the above section.

The use of RNA or DNA based viral systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of nucleic acids could include retroviral, lentivirus, adenoviral, adeno-associated and herpes simplex virus vectors for gene transfer. Viral vectors are currently the most efficient and versatile method of gene transfer in target cells and tissues. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (Sly), human immuno deficiency virus (HIV), and combinations thereof (see, e.g., Buchscher et al., 1992; Johann et al., 1992; Sommerfelt et al., 1990; Wilson et al., 1989; Miller et al., 1999; and PCT/US94/05700).

In applications where transient expression of the nucleic acid is preferred, adenoviral based systems are typically used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors are also used to transduce cells with target nucleic acids, e.g., in the in vitro production of nucleic acids and peptides, and for in vivo and ex vivo gene therapy procedures (see, e.g., West et al., 1987; U.S. Pat. No. 4,797,368; WO 93/24641; Kotin, 1994; Muzyczka, 1994). Construction of recombinant AAV vectors is described in a number of publications, including U.S. Pat. No. 5,173,414; Tratschin et al., 1985; Tratschin, et al., 1984; Hermonat & Muzyczka, 1984; and Samulski et al., 1989.

In particular, numerous viral vector approaches are currently available for gene transfer in clinical trials, with retroviral vectors by far the most frequently used system. All of these viral vectors utilize approaches that involve complementation of defective vectors by genes inserted into helper cell lines to generate the transducing agent. pLASN and MFG-S are examples are retroviral vectors that have been used in clinical trials (Dunbar et al., 1995; Kohn et al., 1995; Malech et al., 1997). PA317/pLASN was the first therapeutic vector used in a gene therapy trial (Blaese et al., 1995). Transduction efficiencies of 50% or greater have been observed for MFG-S packaged vectors (Ellem et al., 1997; and Dranoff et al., 1997).

Recombinant adeno-associated virus vectors (rAAV) are a promising alternative gene delivery systems based on the defective and nonpathogenic parvovirus adeno-associated type 2 virus. All vectors are derived from a plasmid that retains only the AAV 145 by inverted terminal repeats flanking the transgene expression cassette. Efficient gene transfer and stable transgene delivery due to integration into the genomes of the transduced cell are key features for this vector system (Wagner et al., 1998, Kearns et al., 1996).

Replication-deficient recombinant adenoviral vectors (Ad) are predominantly used in transient expression gene therapy; because they can be produced at high titer and they readily infect a number of different cell types. Most adenovirus vectors are engineered such that a transgene replaces the Ad E1a, E1b, and E3 genes; subsequently the replication defector vector is propagated in human 293 cells that supply the deleted gene function in trans. Ad vectors can transduce multiple types of tissues in vivo, including nondividing, differentiated cells such as those found in the liver, kidney and muscle tissues. Conventional Ad vectors have a large carrying capacity. An example of the use of an Ad vector in a clinical trial involved polynucleotide therapy for antitumor immunization with intramuscular injection (Sterman et al., 1998). Additional examples of the use of adenovirus vectors for gene transfer in clinical trials include Rosenecker et al., 1996; Sterman et al., 1998; Welsh et al., 1995; Alvarez et al., 1997; Topf et al., 1998.

Packaging cells are used to form virus particles that are capable of infecting a host cell. Such cells include 293 cells, which package adenovirus, and ψ2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host, other viral sequences being replaced by an expression cassette for the protein to be expressed. The missing viral functions are supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In many gene therapy applications, it is desirable that the gene therapy vector be delivered with a high degree of specificity to a particular tissue type. A viral vector is typically modified to have specificity for a given cell type by expressing a ligand as a fusion protein with a viral coat protein on the viruses outer surface. The ligand is chosen to have affinity for a receptor known to be present on the cell type of interest. For example, Han et al., 1995, reported that Moloney murine leukemia virus can be modified to express human heregulin fused to gp70, and the recombinant virus infects certain human breast cancer cells expressing human epidermal growth factor receptor. This principle can be extended to other pairs of viruses expressing a ligand fusion protein and target cells expressing a receptor. For example, filamentous phage can be engineered to display antibody fragments (e.g., Fab or Fv) having specific binding affinity for virtually any chosen cellular receptor. Although the above description applies primarily to viral vectors, the same principles can be applied to nonviral vectors. Such vectors can be engineered to contain specific uptake sequences thought to favor uptake by specific target cells.

Gene therapy vectors can be delivered in vivo by administration to an individual patient, typically by systemic administration (e.g., intravenous, intraperitoneal, intramuscular, subdermal, or intracranial infusion) or topical application. Alternatively, vectors can be delivered to cells ex vivo, such as cells explanted from an individual patient (e.g., lymphocytes, bone marrow aspirates, and tissue biopsy) or universal donor hematopoietic stem cells, followed by reimplantation of the cells into a patient, usually after selection for cells which have incorporated the vector.

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with a nucleic acid (gene or cDNA), and re-infused back into the subject organism (e.g., patient). Various cell types suitable for ex vivo transfection are well known to those of skill in the art (see, e.g., Freshney et al., 1994; and the references cited therein for a discussion of how to isolate and culture cells from patients).

In one embodiment, stem cells are used in ex vivo procedures for cell transfection and gene therapy. The advantage to using stem cells is that they can be differentiated into other cell types in vitro, or can be introduced into a mammal (such as the donor of the cells) where they will engraft in the bone marrow. Methods for differentiating CD34+ cells in vitro into clinically important immune cell types using cytokines such a GM-CSF, IFN-γ and TNF-α are known (see Inaba et al., 1992).

Stem cells are isolated for transduction and differentiation using known methods. For example, stem cells are isolated from bone marrow cells by panning the bone marrow cells with antibodies which bind unwanted cells, such as CD4+ and CD8+ (T cells), CD45+ (panB cells), GR-1 (granulocytes), and lad (differentiated antigen presenting cells).

Vectors (e.g., retroviruses, adenoviruses, liposomes, etc.) containing therapeutic nucleic acids can be also administered directly to the organism for transduction of cells in vivo. Alternatively, naked DNA can be administered.

Administration is by any of the routes normally used for introducing a molecule into ultimate contact with blood or tissue cells, as described above. The nucleic acids from Tables 2-4 are administered in any suitable manner, preferably with the pharmaceutically acceptable carriers described above. Suitable methods of administering such nucleic acids are available and well known to those of skill in the art, and, although more than one route can be used to administer a particular composition, a particular route can often provide a more immediate and more effective reaction than another route (see Samulski at al., 1989). The present invention is not limited to any method of administering such nucleic acids, but preferentially uses the methods described herein.

The present invention further provides other methods of treating IBD (e.g. Crohn's disease) such as administering to an individual having IBD (e.g. Crohn's disease) an effective amount of an agent that regulates the expression, activity or physical state of at least one gene from Tables 2-4. An “effective amount” of an agent is an amount that modulates a level of expression or activity of a gene from Tables 2-4, in a cell in the individual at least about 10%, at least about 20%, at least about 30%, at least about 40%, at least about 50%, at least about 60%, at least about 70%, at least about 80% or more, compared to a level of the respective gene from Tables 2-4 in a cell in the individual in the absence of the compound. The preventive or therapeutic agents of the present invention may be administered, either orally or parenterally, systemically or locally. For example, intravenous injection such as drip infusion, intramuscular injection, intraperitoneal injection, subcutaneous injection, suppositories, intestinal lavage, oral enteric coated tablets, and the like can be selected, and the method of administration may be chosen, as appropriate, depending on the age and the conditions of the patient. The effective dosage is chosen from the range of 0.01 mg to 100 mg per kg of body weight per administration. Alternatively, the dosage in the range of 1 to 1000 mg, preferably 5 to 50 mg per patient may be chosen. The therapeutic efficacy of the treatment may be monitored by observing various parts of the digestive system and other body parts, or any other monitoring methods known in the art. Other ways of monitoring efficacy can be, but are not limited to monitoring prevention or improvement of diarrhea, prevention or improvement of weight loss, inhibition of bowel tissue edema, inhibition of cell infiltration, inhibition of surviving period shortening, and the like, or any other IBD (e.g. Crohn's disease) related symptom.

The present invention further provides a method of treating an individual clinically diagnosed with IBD (e.g. Crohn's disease). The methods generally comprises analyzing a biological sample that includes a cell, in some cases, a cell, from an individual clinically diagnosed with IBD (e.g. Crohn's disease) for the presence of modified levels of expression of at least 1 gene, at least 10 genes, at least 50 genes, at least 100 genes, or at least 200 genes from Tables 2-4. A treatment plan that is most effective for individuals clinically diagnosed as having a condition associated with IBD (e.g. Crohn's disease) is then selected on the basis of the detected expression of such genes in a cell. Treatment may include administering a composition that includes an agent that modulates the expression or activity of a protein from Tables 2-4 in the cell. Information obtained as described in the methods above can also be used to predict the response of the individual to a particular agent. Thus, the invention further provides a method for predicting a patient's likelihood to respond to a drug treatment for a condition associated with IBD (e.g. Crohn's disease), comprising determining whether modified levels of a gene from Tables 2-4 is present in a cell, wherein the presence of protein is predictive of the patient's likelihood to respond to a drug treatment for the condition. Examples of the prevention or improvement of symptoms accompanied by IBD (e.g. Crohn's disease) that can monitored for effectiveness include prevention or improvement of diarrhea, prevention or improvement of weight loss, inhibition of bowel tissue edema, inhibition of cell infiltration, inhibition of surviving period shortening, and the like, and as a result, or any other IBD (e.g. Crohn's disease) related symptom.

The invention also provides a method of predicting a response to therapy in a subject having IBD (e.g. Crohn's disease) by determining the presence or absence in the subject of one or more markers associated with IBD (e.g. Crohn's disease) described in Tables 5.1, 5.2, 6.2 and 6.3, diagnosing the subject in which the one or more markers are present as having IBD (e.g. Crohn's disease), and predicting a response to a therapy based on the diagnosis e.g., response to therapy may include an efficacious response and/or one or more adverse events. The invention also provides a method of optimizing therapy in a subject having IBD (e.g. Crohn's disease) by determining the presence or absence in the subject of one or more markers associated with a clinical subtype of IBD (e.g. Crohn's disease), diagnosing the subject in which the one or more markers are present as having a particular clinical subtype of IBD (e.g. Crohn's disease), and treating the subject having a particular clinical subtype of IBD (e.g. Crohn's disease) based on the diagnosis. As an example, treatment for the fibrostenotic subtype of Crohn's disease currently includes surgical removal of the affected, structured part of the bowel.

Thus, while there are a number of treatments for Crohn's disease currently available, they all are accompanied by various side effects, high costs, and long complicated treatment protocols, which are often not available and effective in a large number of individuals. Accordingly, there remains a need in the art for more effective and otherwise improved methods for treating and preventing Crohn's disease. Thus, there is a continuing need in the medical arts for genetic markers of IBD (e.g. Crohn's disease) and guidance for the use of such markers. The present invention fulfills this need and provides further related advantages.

### EXAMPLES

### Example 1

### Identification of Cases and Controls

Samples from two human populations were used for this study: Quebec Founder and German populations. The text below describes the details for the recruitment of the individuals from those two populations and used for the study's genetic analysis.

All Quebec Founder individuals were sampled from the Quebec founder population (QFP). Membership in the founder population was defined as having four grandparents with French Canadian family names who were born in the Province of Quebec, Canada or in adjacent areas of the Provinces of New Brunswick and Ontario or in New England or New York State. The Quebec population is characterized both by extended LD and by decreased genetic heterogeneity. The increased extent of LD allows the detection of disease associated genes using a reasonable marker density, while still allowing the increased meiotic resolution of population-based mapping. The specific combination of age in generations, optimal number of founders and large present population size makes the QFP optimal for LD-based gene mapping.

Patient inclusion criteria for the QFP study include diagnosis for Crohn's disease by any one of the following: a colonoscopy, a radiological examination with barium, an abdominal surgical operation or a biopsy or a surgical specimen. The colonoscopy diagnosis consists of observing linear, deep or serpentigenous ulcers, pseudopolyps, or skip areas. The barium radiological examination consists of the detection of strictures, ulcerations and string signs by observing the barium enema and the small bowel followed through an NMRI series.

Patients that were diagnosed with ulcerative colitis, infectious colitis or other intestinal diseases were excluded from the study. All human sampling was subject to ethical review procedures.

All enrolled QFP subjects (patients and controls) provided a 30 ml blood sample (3 barcoded tubes of 10 ml). Samples were processed immediately upon arrival at Genizon's laboratory. All samples were scanned and logged into a LabVantage Laboratory Information Management System (LIMS), which served as a hub between the clinical data management system and the genetic analysis system. Following centrifugation, the buffy coat containing the white blood cells was isolated from each tube. Genomic DNA was extracted from the buffy coat from one of the tubes, and stored at 4° C. until required for genotyping. DNA extraction was performed with a commercial kit using a guanidine hydrochloride based method (FlexiGene, Qiagen) according to the manufacturer's instructions. The extraction method yielded high molecular weight DNA, and the quality of every DNA sample was verified by agarose gel electrophoresis. Genomic DNA appeared on the gel as a large band of very high molecular weight. The remaining two buffy coats were stored at −80° C. as backups.

The QFP samples were collected as family trios consisting of Crohn's disease subjects and two first degree relatives. Of the 500 trios, 477 were Parent, Parent, Child (PPC) trios; the remainders were Parent, Child, Child (PCC) trios. Only the PPC trios were used for the analysis reported here because they produced equal numbers of more accurately estimated case and control haplotypes than the PCC trios. 382 trios were used in the genome wide scan. One member of each trio was affected with Crohn's disease. For the 382 trios used in the genome wide scan, these included 189 daughters, 90 sons, 54 mothers and 49 fathers. When a child was the affected member of the trio, the two non-transmitted parental chromosomes (one from each parent) were used as controls, when one of the parents was affected, that person's spouse provided the control chromosomes. The recruitment of trios allowed a more precise determination of long extended haplotypes.

The German patients (cases) were recruited at the Charité University Hospital (Berlin, Germany) and the Department of General Internal Medicine of the Christian-Albrechts-University (Kiel, Germany), with the support of the German Crohn and Colitis Foundation. Clinical, radiological and endoscopic (i.e. type and distribution of lesions) examinations were required to unequivocally confirm the diagnosis of Crohn disease, and histological findings also had to be confirmative of, or compatible with, the diagnosis. In case of uncertainty, patients were excluded from the study. The patient sample has been used in several studies before and the respective publications provide a more detailed account of the phenotyping techniques employed. German control individuals were obtained from the POPGEN biobank. All recruitment protocols were approved by ethics committees at the participating centres prior to commencement of the study and participants were obliged to give written, informed consent. The German samples were collected as cases and controls consisting of Crohn disease subjects and controls. A total of 493 cases and 493 controls were collected for this study.

### Example 2

### Genotyping

### QFP Samples Genotyping

Genotyping was performed using Perlegen's ultra-high-throughput platform. Marker loci were amplified by PCR and hybridized to wafers containing arrays of oligonucleotides. Allele discrimination was performed through allele-specific hybridization. In total, 248,535 SNPs, distributed as evenly as possible throughout the genome, were genotyped on the 382 QFP trios for a total of 372,802,500 genotypes. These markers were mostly selected from various databases including the ˜1.6 million SNP database of Perlegen Life Sciences (Patil, 2001); several thousand were obtained from the HapMap consortium database and/or dbSNP at NCBI. The SNPs were chosen to maximize uniformity of genetic coverage and to cover a distribution of allele frequencies. All SNPs that did not pass the quality controls for the assay, that is, that had a minor allele frequency of less than 1%, a Mendelian error rate within trios greater than 1%, that deviated significantly from the Hardy-Weinberg equilibrium, or that had excessive missing data (cut-off at 1% missing values or higher) were removed from the analysis. Genetic analysis was performed on a total of 165,803 SNPs (158,811 autosomal, 6851 X chromosome and 141 Y chromosome). The average gap size was approximately 17 kb. Of the 165,803 markers, ˜140,000 had a minor allele frequency (MAF) greater than 10% for the QFP.

### German Samples Genotyping

Genotyping was performed by Illumina, using Illumina's HumanHap 550 Genotyping Beadchip and standard technologies. The HumanHap-550 chip includes over 550,000 tag SNPs derived from the International HapMap project. The Illumina BeadStudio software was used to perform clustering for genotype assessment. The genotyping information was entered into a Unified Genotype Database from which it was accessed using custom-built programs for export to the genetic analysis pipeline. Analyses of these genotypes were performed with the statistical tools described in Example 3.

### Example 3

### Genetic Analysis

1. Dataset Quality Assessment

### 1.1 Related Individuals

The application PLINK was run to identify and remove identical or closely related individuals (i.e. proportion of alleles identical-by-state given as pi-hat≧0.4581) within the dataset.

### 1.2 Stratification

Prior to performing genetic analyses, the complete set of cases and controls was assessed for population substructure. Population substructure, or stratification, refers to a difference in allele frequencies between cases and controls that is not due to true disease association but due to other factors, e.g. ethnic differences in genetic background.

The program StratFinder was used to test for stratification.

### 1.3 Cleaning

The data were then subjected to QC by performing a cleaning step Using the program DataStets, which calculates the following statistics per marker or per <individual>:
Minor allele frequency (MAF) for each marker
Number of markers with MAF <5%, <4%, <3%, <2%, <1%
Number of missing values for each marker and individual
Monomorphic markers
Departure from Hardy-Weinberg equilibrium within control individuals for each marker

The following acceptance criteria were required for further analysis:
Missing values per marker or individual <1%
Minor allele frequency per marker ≧4%,
Allele frequencies for controls in Hardy-Weinberg equilibrium (cut-off=Log₁₀(0.05/# of markers tested per chromosome)

Markers and individuals not meeting these criteria were removed from the dataset using DataPullPC.

2. Phase Determination

Haplotypes were estimated from the case/control genotype data using ggplem, a modified version of the PL-EM algorithm. The programs geno2patctr and tagger determined case and control genotypes and prepared the data in the input format for PL-EM. An EM algorithm module consisting of several applications was used to resolve phase ambiguities. PLEMPre first recoded the genotypes for input into the PL-EM algorithm which used an 11-marker sliding block for haplotype estimation and deposited the constructed haplotypes into a file, happatctr which was the input file for haplotype association analysis performed by the program, LDSTATS.

The program GeneWriter was used to create a case-control genotype file, genopatctr, which was the input for the program, SINGLETYPE, which was used to perform single marker case-control association analysis.

3. Haplotype Association Analysis

Haplotype association analysis was performed using the program LDSTATS. LDSTATS tests for association of haplotypes with the disease phenotype. The algorithm LDSTATS (v2.0) defines haplotypes using multi-marker windows that advance across the marker map in one-marker increments. Windows of size 1, 3, 5, 7, and 9 were analyzed. At each position the frequency of haplotypes in cases and controls was determined and a chi-square statistic was calculated from case control frequency tables. For LDSTATS v2.0, the significance of the chi-square for single marker and 3-marker windows was calculated as Pearson's chi-square with degrees of freedom. Larger windows of multi-allelic haplotype association were tested using Smith's normalization of the square root of Pearson's Chi-square. LDSTATS v4.0 calculates significance of chi-square values using a permutation test in which case-control status is randomly permuted until 350 permuted chi-square values are observed that are greater than or equal to chi-square value of the actual data. The P value is then calculated as 350/the number of permutations required.

Table 5.1 lists the results for association analysis using LDSTATs (v2.0 and v4.0) for the 371 regions described above based on the genome wide scan genotype data for 382 QFP trios. For each region that was associated with Crohn disease in the genome wide scan, we report in Table 5.2 the allele frequencies and the relative risk (RR) for the haplotypes contributing to the best signal at each SNP in the region. The best signal at a given location was determined by comparing the significance (p-value) of the association with Crohn disease for window sizes of 1, 3, 5, 7, and 9 SNPs, and selecting the most significant window. For a given window size at a given location, the association with Crohn disease was evaluated by comparing the overall distribution of haplotypes in the cases with the overall distribution of haplotypes in the controls. Haplotypes with a relative risk greater than one increase the risk of developing Crohn disease while haplotypes with a relative risk less than one are protective and decrease the risk.

Tables 6.1 and 6.2 list the results for association analysis using LDSTATs (v2.0) for the candidate regions described in Table 1 based on the genome wide scan genotype data for the German cases and controls. For each one of these regions, we report in Tables 6.3 the allele frequencies and the relative risk (RR) for the haplotypes contributing to the best signal at each SNP in the region. For clarity purposes, Table 6.1 contains genetic markers that are not part of the invention, but are necessary for understanding the invention. The invention tables are Tables 5.1, 5.2, 6.2 and 6.3.

### Example 4

### Gene Identification and Characterization

A series of gene characterization was performed for each candidate region described in Table 1. Any gene or EST mapping to the interval based on public map data or proprietary map data was considered as a candidate CROHN disease gene. The approach used to identify all genes located in the critical regions is described below.

Public Gene Mining

Once regions were identified using the analyses described above, a series of public data mining efforts were undertaken, with the aim of identifying all genes located within the critical intervals as well as their respective structural elements (i.e., promoters and other regulatory elements, UTRs, exons and splice sites). The initial analysis relied on annotation information stored in public databases (e.g. NCBI, UCSC Genome Bioinformatics, Entrez Human Genome Browser, OMIM—see below for database URL information). Table 2 lists the genes that have been mapped to the candidate regions.

For some genes the available public annotation was extensive, whereas for others very little was known about a gene's function. Customized analysis was therefore performed to characterize genes that corresponded to this latter class. Importantly, the presence of rare splice variants and artifactual ESTs was carefully evaluated. Subsequent cluster analysis of novel ESTs provided an indication of additional gene content in some cases. The resulting clusters were graphically displayed against the genomic sequence, providing indications of separate clusters that may contribute to the same gene, thereby facilitating development of confirmatory experiments in the laboratory. While much of this information was available in the public domain, the customized analysis performed revealed additional information not immediately apparent from the public genome browsers.

A unique consensus sequence was constructed for each splice variant and a trained reviewer assessed each alignment. This assessment included examination of all putative splice junctions for consensus splice donor/acceptor sequences, putative start codons, consensus Kozak sequences and upstream in-frame stops, and the location of polyadenylation signals. In addition, conserved noncoding sequences (CNSs) that could potentially be involved in regulatory functions were included as important information for each gene. The genomic reference and exon sequences were then archived for future reference. A master assembly that included all splice variants, exons and the genomic structure was used in subsequent analyses (i.e., analysis of polymorphisms). Table 3 lists gene clusters based on the publicly available EST and cDNA clustering algorithm, ECGene.

An important component of these efforts was the ability to visualize and store the results of the data mining efforts. A customized version of the highly versatile genome browser GBrowse (http://www.gmod.org/) was implemented in order to permit the visualization of several types of information against the corresponding genomic sequence. In addition, the results of the statistical analyses were plotted against the genomic interval, thereby greatly facilitating focused analysis of gene content.

Computational Analysis of Genes and GeneMaps

In order to assist in the prioritization of candidate genes for which minimal annotation existed, a series of computational analyses were performed that included basic BLAST searches and alignments to identify related genes. In some cases this provided an indication of potential function. In addition, protein domains and motifs were identified that further assisted in the understanding of potential function, as well as predicted cellular localization.

A comprehensive review of the public literature was also performed in order to facilitate identification of information regarding the potential role of candidate genes in the pathophysiology of CROHN disease. In addition to the standard review of the literature, public resources (Medline and other online databases) were also mined for information regarding the involvement of candidate genes in specific signaling pathways. A variety of pathway and yeast two hybrid databases were mined for information regarding protein-protein interactions. These included BIND, MINT, DIP, Interdom, and Reactome, among others. By identifying homologues of genes in the CROHN candidate regions and exploring whether interacting proteins had been identified already, knowledge regarding the GeneMaps for CROHN disease was advanced. The pathway information gained from the use of these resources was also integrated with the literature review efforts, as described above.

All references cited herein are hereby incorporated by references in their entireties.

1. A method of constructing a GeneMap for IBD (e.g. Crohn's Disease) comprising identifying at least two chromosomal loci associated with IBD (e.g. Crohn's Disease), wherein said at least two chromosomal loci are selected from the genomic regions listed in Table 1.

2. The method of claim 1, wherein said population is a general population or a founder population.

3. (canceled)

4. The method of claim 2, wherein said founder population is the German population or the Quebec founder population.

5. The method of claim 1, wherein said at least two chromosomal regions are selected from the genes in Table 2, 3 or 4.

6.-7. (canceled)

8. The method of claim 1, wherein the identifying comprises screening for the presence or absence of at least one single nucleotide polymorphisms (SNPs) from Tables 5.1, 5.2, 6.2 and 6.3 in at least one sample.

9. The method of claim 8, wherein the screening comprises the steps of: (a) obtaining biological samples from at least one disease patient; (b) screening for the presence or absence of at least one SNP or a group of SNPs from Tables 5.1, 5.2, 6.2 and 6.3 within each biological sample; and (c) evaluating whether said SNP or a group of SNPs shows a statistically significant skewed genotype distribution between a group of patients compared to a control.

10. The method of claim 9, wherein said biological samples are selected from the group consisting of fluid, biopsy samples, serum, tissue or buccal swabs, saliva, mucus, urine, stools, vaginal secretions, lymph, amiotic liquid, pleural liquid or tears.

11.-13. (canceled)

14. The method of claim 8, wherein said screening is performed by a method selected from the group consisting of an allele-specific hybridization assay, an oligonucleotide ligation assay, an allele-specific elongation/ligation assay, an allele-specific amplification assay, a single-base extension assay, a molecular inversion probe assay, an invasive cleavage assay, a selective termination assay, RFLP, a sequencing assay, SSCP, a mismatch-cleaving assay, and denaturing gradient gel electrophoresis.

15. The method of claim 8, wherein said screening is carried out on each individual of a cohort at each of at least one SNP or a group of SNPs from Tables 5.1, 5.2, 6.2 and 6.3.

16.-19. (canceled)

20. The method of claim 1, wherein the GeneMap comprises all of the genes of Tables 2-4.

21.-38. (canceled)

39. A method of detecting susceptibility to IBD (e.g. Crohn's Disease) comprising detecting at least one mutation or polymorphism in the nucleic acid molecule selected from Tables 2-4 in a patient.

40. The method of claim 39, wherein said method comprises hybridizing a probe to said patient's sample of DNA or RNA under stringent conditions which allow hybridization of said probe to nucleic acid comprising said mutation or polymorphism, wherein the presence of a hybridization signal indicates the presence of said mutation or polymorphism in at least one gene from Tables 2-4.

41. The method of claim 39, wherein the patient's DNA or RNA has been amplified and said amplified DNA or RNA is hybridized.

42. The method of claim 39, wherein said method comprises using a single-stranded conformation polymorphism technique to assay for said mutation.

43. The method of claim 39, wherein said method comprises sequencing at least one gene from Tables 2-4 in a sample of RNA or DNA from a patient.

44. The method of claim 39, wherein said method comprises determining the sequence of at least one gene from Tables 2-4 by preparing cDNA from RNA taken from said patient and sequencing said cDNA to determine the presence or absence of a mutation.

45. The method of claim 39, wherein said method comprises performing an RNAse assay.

46. The method of claim 39, wherein said probe is attached to a microarray or a bead.

47. The method of claim 39, wherein said probes are oligonucleotides.

48. The method of claim 40, wherein said sample is selected from the group consisting of blood, normal tissue and tumor tissue.

49. The method of claim 39, wherein the mutation is selected from the group consisting of at least one of the SNPs from Tables 5.1, 5.2, 6.2 and 6.3, alone or in combination.

50.-51. (canceled)

52. A method of diagnosing susceptibility to IBD (e.g. Crohn's Disease) in an individual, comprising screening for an at-risk haplotype of at least one gene or gene region from Tables 2-4, that is more frequently present in an individual susceptible to IBD (e.g. Crohn's Disease) compared to a control individual, wherein the presence of the at-risk haplotype is indicative of a susceptibility to IBD (e.g. Crohn's Disease).

53. The method of claim 52 wherein the at-risk haplotype is indicative of increased risk for IBD (e.g. Crohn's Disease).

54. The method of claim 53, wherein the risk is increased at least about 20%.

55. The method of claim 52, wherein the at-risk haplotype is characterized by the presence of at least one single nucleotide polymorphism from Tables 5.1, 5.2, 6.2 and 6.3.

56. The method of claim 52, wherein screening for the presence of an at-risk haplotype in at least one gene from Tables 2-4, comprises enzymatic amplification of nucleic acid from said individual or amplification using universal oligos on elongation/ligation products.

57. The method of claim 56, wherein the nucleic acid is DNA.

58. The method of claim 57, wherein the DNA is human DNA.

59. The method of claim 52, wherein screening for the presence of an at-risk haplotype in at least one gene from Tables 2-4 comprises: (a) obtaining material containing nucleic acid from the individual; (b) amplifying said nucleic acid; and (c) determining the presence or absence of an at-risk haplotype in said amplified nucleic acid.

60. The method of claim 59, wherein determining the presence of an at-risk haplotype is performed by electrophoretic analysis, restriction length polymorphism analysis, sequence analysis or hybridization analysis.

61.-65. (canceled)

66. A drug screening assay comprising: (a) administering a test compound to an animal having IBD (e.g. Crohn's Disease), or a cell population isolated therefrom; and (b) comparing the level of gene expression of at least one gene from Tables 2-4 in the presence of the test compound with the level of said gene expression in normal cells; wherein test compounds which provide the level of expression of one or more genes from Tables 2-4 similar to that of the normal cells are candidates for drugs to treat IBD (e.g. Crohn's Disease).

67.-68. (canceled)

69. A method for predicting the efficacy of a drug for treating IBD (e.g. Crohn's Disease) in a human patient, comprising: (a) obtaining a sample of cells from the patient; (b) obtaining a gene expression profile from the sample in the absence and presence of the drug; the gene expression profile comprising one or more genes from Tables 2-4; and (c) comparing the gene expression profile of the sample with a reference gene expression profile, wherein similarity between the sample expression profile and the reference expression profile predicts the efficacy of the drug for treating IBD (e.g. Crohn's Disease) in the patient.

70. The method of claim 69, further comprising exposing the sample to the drug for treating IBD (e.g. Crohn's Disease) prior to obtaining the gene expression profile of the sample.

71. The method of claim 69, wherein the sample of cells is derived from a tissue selected from the group consisting of: the brain, respiratory system, digestive system, skin, scalp, muscle and nervous tissue.

72. The method of claim 71, wherein the cells are selected from the group consisting of: digestive system cell, colon cell, vaginal cell, hair cell, brain cell, muscle cell, neutrophil, dentric cell, T cell, mast cell, CD4+ lymphocyte, monocyte, macrophage, dendritic cell, and epithelial cell.

73. The method of claim 69, wherein the sample is obtained via biopsy.

74. The method of claim 69, wherein the gene expression profile comprises expression values for all of the genes listed in Tables 2-4.

75. The method of claim 74, wherein the gene expression profile of the sample is obtained by detecting the protein products of said genes.

76. The method of claim 69, wherein the gene expression profile of the sample is obtained using a hybridization assay to oligonucleotides contained in a microarray.

77. The method of claim 76, wherein the oligonucleotides comprises nucleic acid molecules at least 95% identical to the gene sequences from Tables 2-4.

78. The method of claim 69, wherein the reference expression profile is that of cells derived from patients that do not have IBD (e.g. Crohn's Disease).

79. The method of claim 69, wherein the drug is selected from the group consisting of symptom relievers.

80. The method of claim 69, wherein said patient's sample of DNA has been amplified or cloned.

81. A method for predicting the efficacy of a drug for treating IBD (e.g. Crohn's Disease) in a human patient, comprising: (a) obtaining a sample of cells from the patient; (b) obtaining a set of genotypes from the sample, wherein the set of genotypes comprises genotypes of one or more polymorphic loci from Tables 2-4, 5.1. 5.2, 6.2, 6.3; and (c) comparing the set of genotypes of the sample with a set of genotypes associated with efficacy of the drug, wherein similarity between the set of genotypes of the sample and the set of genotypes associated with efficacy of the drug predicts the efficacy of the drug for treating IBD (e.g. Crohn's Disease) in the patient.

82. The method of claim 81, wherein the sample of cells is derived from a tissue selected from the group consisting of: colon cell, vaginal cell, skin, brain, nervous system, digestive system, respiratory system, and scalp.

83. The method of claim 82, wherein the cells are selected from the group consisting of: digestive system cell, hair cell, brain cell, muscle cell, neutrophil, dentric cell, T cell, mast cell, CD4+ lymphocyte, monocyte, macrophage, dendritic cell, and epithelial cell.

84. The method of claim 81, wherein the sample is obtained via biopsy.

85. The method of claim 81, wherein the set of genotypes from the sample comprises genotypes of at least two of the polymorphic loci listed in Tables 2-4, 5.1. 5.2, 6.2, 6.3.

86. The method of claim 81 wherein the set of genotypes from the sample is obtained by hybridization to allele-specific oligonucleotides complementary to the polymorphic loci from Tables 2-4, 5.1. 5.2, 6.2, 6.3, wherein said allele-specific oligonucleotides are contained on a microarray.

87. The method of claim 86, wherein the oligonucleotides comprise nucleic acid molecules at least 95% identical to SEQ ID from Tables 2-4, 5.1. 5.2, 6.2, 6.3.

88. The method of claim 81 wherein the set of genotypes from the sample is obtained by sequencing said polymorphic loci in said sample.

89. The method of claim 81, wherein the drug is selected from the group consisting of symptom relievers and drugs for IBD (e.g. Crohn's Disease).

90.-117. (canceled)

118. A method for identifying a gene that regulates drug response in IBD (e.g. Crohn's Disease), comprising: (a) obtaining a gene expression profile for at least one gene from Tables 2-4 in a resident tissue cell induced for a proinflammatory like state in the presence of the candidate drug; and (b) comparing the expression profile of said gene to a reference expression profile for said gene in a cell induced for the pro-inflammatory like state in the absence of the candidate drug, wherein genes whose expression relative to the reference expression profile is altered by the drug may identifies the gene as a gene that regulates drug response in IBD (e.g. Crohn's Disease).

119.-125. (canceled)

126. A kit for assessing a patient's risk of having or developing IBD (e.g. Crohn's Disease), comprising: (a) a detection means for detecting the genotype of at least one polymorphic locus shown in Tables 2-4, 5.1. 5.2, 6.2, 6.3; and (b) instructions for correlating the genotype of said at least one polymorphic locus with a patient's risk of having or developing IBD (e.g. Crohn's Disease).

127. The kit of claim 126, wherein the detection means includes nucleic acid probes for detecting the genotype of said at least one polymorphic locus.

128.-137. (canceled)

138. A method of assessing a patient's risk of having or developing IBD (e.g. Crohn's Disease), comprising (a) determining a genotype for at least one polymorphic locus from Tables 2-4, 5.1. 5.2, 6.2, 6.3 in a patient; (b) comparing said genotype of (a) to a genotype for at least one polymorphic locus from Tables 2-4, 5.1. 5.2, 6.2, 6.3 that is associated with IBD (e.g. Crohn's Disease); and (c) assessing the patient's risk of having or developing IBD (e.g. Crohn's Disease), wherein said patient has a higher risk of having or developing IBD (e.g. Crohn's Disease) if the genotype for at least one polymorphic locus from Tables 2-4, 5.1. 5. 2, 6.2, 6.3 in said patient is the same as said genotype for at least one polymorphic locus from Tables 2-4, 5.1. 5.2, 6.2, 6.3 that is associated with IBD (e.g. Crohn's Disease).

139.-140. (canceled)
